(19) United States
(12) Patent Application Publication
(10) Pub. No.: US 2016/0168210 A1
(43) Pub. Date: Jun. 16, 2016

(54) DERIVATIVES OF COLLAGEN-BINDING HAIRPIN PEPTIDES

(71) Applicant: NATIONAL RESEARCH COUNCIL OF CANADA, Ottawa (CA)

(72) Inventors: Feng NI, Pierrefonds (CA); Ping XU, St-Laurent (CA); Sazzad HOSSAIN, Pointe-Claire (QC); Dmitri TOLKATCHEV, La Prairie (CA); Louis-Philippe RICHER, Ville St-Laurent (CA)

(73) Assignee: NATIONAL RESEARCH COUNCIL OF CANADA, Ottawa, ON (CA)

(21) Appl. No.: 14/436,597

(22) PCT Filed: Oct. 18, 2013
(86) PCT No.: PCT/CA2013/000894

§ 371 Date: Apr. 17, 2015

Related U.S. Application Data
(60) Provisional application No. 61/716,093, filed on Oct. 19, 2012.

(51) Int. Cl. C07K 14/435

(52) U.S. Cl. CPC C07K 14/435; A61K 38/00

(57) ABSTRACT

Compounds of interest, for example active pharmaceutical ingredients, probes or inactive carriers, may be delivered to a site of interest by conjugating the compound of interest to a collagen-binding linear hairpin (CBLH) peptide to form a molecule of Formula (I) and then providing the molecule to the site of interest where the CBLH peptide binds to collagen at the site of interest thereby delivering the compound of interest to the site of interest. Molecule of Formula (I) are: (I) where: Y is a first compound of interest; Z is a second compound of interest; n and m are independently 0 or 1 with the proviso that at least one of n and m is 1; and, CBLH is a collagen-binding linear hairpin peptide having 19 or fewer amino acids and having a turn amino acid sequence comprising 4 to 6 amino acid residues providing a stable turn structure, the turn sequence flanked on one side by a first flanking sequence having a WT(W/Y) motif and flanked on the other side by a RR second flanking sequence having a WT(W/R) motif, the W residue at position 1 of the first flanking sequence forming a cross-strand indole-indole or cation-π interaction pair with the amino acid residue at position 3 of the second flanking sequence without any disulfide bond.

### FIELD OF THE INVENTION

The present invention relates to derivatives of collagen-binding hairpin peptides, particularly peptides for imaging and drug delivery to fibrosing/fibrotic tissues.

### BACKGROUND OF THE INVENTION

Targeted delivery is an actively sought-after strategy for the control of pharmaceutical action of drugs that are either too toxic for long-term administration or chemically unstable, leading to reduced efficacy. Targeted drug delivery employs the conjugation of a small-molecule drug or a protein therapeutic with a targeting moiety such as a polypeptide, an antibody or a polymer to achieve drug accumulation at sites of pathology (Duncan 2003; Majumdar 2012). The pharmaceutical action is thereby concentrated at local tissues and/or activated (or released) by unique enzymes or changes of the physicochemical environment, e.g. for anti-thrombotic drug delivery (Peter 2003; Topcic 2011) or for anti-cancer therapies (Rooseboom 2004).

The requirement for targeted therapies is the availability of targeting moieties (TM) specific for biomarkers of disease-causing cells and/or pathological tissues. In addition to targeting specificity, the TM-biomarker pair must also accumulate in diseased tissues in sufficient concentrations, which may not be possible to achieve due to the often-observed spatiotemporal variations of biomarkers across different tissue locations. The latest research has therefore placed emphasis on the use of peptide ligands specific for components of the extracellular matrix (ECM), especially those that are either unique or exposed in pathological tissues (Rothenfluh 2008; O'Neil 2009; Peters 2009; Muzzard 2009; Chan 2010). Fibrotic/fibrosing tissues are of particular interest in this regard, since fibroproliferative diseases caused by ECM degradation and fibrotic scarring are the underlining causes of lethality associated with many chronic ailments of the heart, kidney, liver, lungs, joints, the skin or the vasculature in general (Wynn 2004). As such, there has been a constant search for more effective and tissue-specific targeting agents to facilitate disease diagnosis and localized drug delivery (Caravan 2007; Rothenfluh 2008; O'Neil 2009; Peters 2009; Muzzard 2009; Helms 2009; Chan 2010).

Published work up to date (Table 1) has reported short peptides ranging from 5-10 residues as specific binders for abundant components of the extracellular matrix, especially fibrin or collagens (Takagi 1992; Vanhoorelbeke 2003; Rothenfluh 2008; Peters 2009; Helms 2009; Chan 2010; Sawada 2011). Some longer peptides (of 12-13 residues) in cyclized forms have been found as specific binders for the most abundant ECM protein, type-I collagen (Caravan 2007; Muzzard 2009). However, these long peptides need to be modified to include Cys residues at locations suitable for cyclization and affinity enhancement. Generally, practical applications of peptide-based targeting agents are often limited by conformational flexibility and a lack of well-defined secondary and tertiary structures of short linear peptides (see, for example, Collier 2011). Introduction of disulfide-forming Cys residues will, on the other hand, make it more difficult to incorporate the collagen-binding functionality into bioactive proteins, e.g. for collagen-based delivery of cytokines or growth factors (Han 2009, Sun 2009).

TABLE 1
List of selected collagen-binding peptides reported in literature

| Name | Source | Sequence | |
|---|---|---|---|
| III-3 | Takagi 1992 | WREPSFCALS | (SEQ ID NO: 42) |
| Q-peptide | Depraetere 1998 | CVWLWEQC | (SEQ ID NO: 43) |
| N-peptide | Depraetere 1998 | CVWLWENC | (SEQ ID NO: 44) |
| C6H5 | Vanhoorelbeke 2003 | CMTSPWRC | (SEQ ID NO: 45) |
| C6G12 | Vanhoorelbeke 2003 | CRTSPWRC | (SEQ ID NO: 46) |
| C6Al2 | Vanhoorelbeke 2003 | CYRSPWRC | (SEQ ID NO: 47) |
| EP-3533p | Caravan 2007 | GKWHCTTKFPHHYCLY | (SEQ ID NO: 48) |
| C1-3 | Rothenfluh 2008 | WYRGRL | (SEQ ID NO: 49) |
| | Helms 2009 | HVWMQAP | (SEQ ID NO: 50) |
| | Muzzard 2009 | CPGRVMHGLHLGDDEGPC | (SEQ ID NO: 51) |
| C-11 | Chan 2010 | KLWLLPK | (SEQ ID NO: 52) |

Rothenfluh et al (2008) described a 6-residue peptide, WYRGRL, discovered using a phage-displayed library panned against collagen IIα1, a locally-enriched component of the cartilage matrix. A peptide-nanoparticle conjugate was prepared using an acetylated and Cys-modified peptide Ac-WYRGRLC and thiol-reactive conjugation chemistry. The peptide-polymer nanoparticle, WYRGRL-polypropylene sulphide (or PPS), was shown to target and bind to articular cartilage tissue as promising drug delivery vehicles (Setton 2008).

In another publication, a 6-residue peptide KLWLLPK was reported as a specific binder of collagen IV, which is a main component of the vascular basement membrane (Chan 2010). A peptide-conjugated nanoparticule system, called nanoburrs, was prepared from a modified peptide KLWLLPKGGC using thiol-maleimide conjugation chemistry. This peptide-conjugated nanoparticle was shown to enable spatiotemporal controlled delivery to injured vasculatures.

Phage display was also used to discover peptides that bind to tissue grafts composed of chondroitin sulfate and collagen for drug-delivery applications (Sawada, 2011). Some of these phage-derived peptides contained no cysteine residues nor disulfide bonds and were highly enriched with Trp residues. However, none of these peptides was shown to specifically bind collagen or to other components in the artificial tissue graft used for phage panning.

The possibility of targeting the most abundant extracellular protein, type-I collagen, has been demonstrated in vivo by Caravan et al (2007), by Helms et al (2009) and by Muzzard et al (2009) using animal models of various pathological conditions. In all these studies, it was necessary to employ affinity enhancement strategies such as disulfide-mediated cyclization, as in the cyclic peptide moieties KWHCTTKFPHHYCLY (Caravan 2007) and CPGRVMHG-LHLGDDEGPC (Muzzard 2009) or the multivalent conjugation of the linear peptide HVWMQAPGGGK to synthetic dendrimers (Helms 2009).

Earlier work relied on identifying short peptides that can mimic the binding of von Willebrand factor (vWF) to type-I collagen (Takagi 1992; Depraetere 1998; Vanhoorelbake 2003). Therefore, Takagi et al (Takagi 1992) reported a 10-residue peptide fragment, WREPSFCALS, derived from vWF as having the capacities to bind collagen and inhibit the vWF-collagen interaction. Using phage display, Depraetere et al (Depraetere 1998) isolated two 6-residue cyclized peptides, CVWLWEQC and CVWLWENC, as epitopes potentially mimicking vWF, and inhibiting the interactions of vWF with type-I collagen. In a more recent study, the same research group identified a consensus sequence SPWR potentially mimicking a discrete epitope in vWF in the form of representative cyclic peptides, CMTSPWRC, CRTSPWRC and CYRSPWRC (Vanhorredbeke 2003).

Despite attempts at discovery and design of collagen-binding peptides (Table 1), none of the above collagen-specific compounds is known to bind different states of collagen, especially the monomeric versus the more prevalent polymerized collagen fibrils. Regardless, one striking feature of almost all the above peptide ligands, except that of Caravan et al (2007), is the presence of tryptophan residues as one potential determinant for specific binding to collagen. Another important characteristic is the need for peptide cyclization, mostly in the form of a disulfide bond, which reduces the conformational flexibility of linear peptide fragments and which in certain cases may induce the formation of unique three-dimensional structures required for binding specificity.

From a purely structural point of view, cyclization of linear peptides has been shown to invariably induce the formation of a (β)-turn structure and in some cases well-folded β-hairpin structures emerge from cyclic peptides with certain sequence characteristics (Cochran 2001a; Mirassou 2009). A recent study reported that non-covalent indole-indole interactions, as conferred by a Trp-Trp pair, can largely reproduce the native hairpin structure of a bioactive peptide (Mirassou 2009). Such a β-hairpin structure stabilized by a Trp-Trp pair is also known for a class of linear peptides that contain additional Trp residues, referred to as tryptophan zippers or trpzip peptides (Cochran 2001b, Cochran 2005; Cochran 2007). It is not yet known, however, that any such Trp-rich linear hairpin peptide has a binding capacity for abundant components of the ECM, especially not for collagens.

Trpzip peptides, especially trpzip1, trpzip4, trpzip5 and trpzip6 (Cochran 2001b) were employed as heat-sensitive linkers to enable the control of the inhibitory activities of a new class of bivalent thrombin inhibitors (WO/2012/142696). Trpzip linkers in heat-sensitive bivalent thrombin-inhibitors were found to respond to the presence of type-I collagen, the most abundant extracellular protein particularly enriched in inflamed vascular lesions. It was further disclosed that responsiveness to collagen resides within the trpzip linker segments and that the trpzip peptides alone, also respond to type I collagen. A series of new NMR-based binding data revealed that all the trpzip peptides and bivalent thrombin inhibitors containing these hairpin peptides studied respond to and appear to have specific affinity for only unpolymerized or monomeric collagen abundant in fibrotic/fibrosing tissues undergoing active and uncontrolled remodelling. Most importantly, localization of a representative compound in diseased (fibrotic) tissues was determined through fluorescence imaging using both a rat model of venous thrombosis and a mouse model of pulmonary fibrosis. Such biophysical (NMR) and in vivo data show that compounds bearing a collagen-binding linear hairpin have an affinity for inflamed/fibrosing tissues in vivo, very likely due to specific binding to newly-secreted collagens enriched in these tissue environments.

### SUMMARY OF THE INVENTION

The present invention relates to derivatives of collagen-binding hairpin peptides, particularly peptides for imaging and drug delivery to fibrosing/fibrotic tissues.

The present invention provides a molecule of Formula (I)

(Y)ₙ-(CBLH)-(Z)ₘ (I)

that specifically binds to collagen. In the molecule as described above, Y is a first compound of interest; Z is a second compound of interest; Y and Z may be different or the same, and n and m are independently 0 or 1. At least one of n and m is 1. Additionally, CBLH is a collagen-binding linear hairpin peptide comprising 19 or fewer amino acids and comprising a turn amino acid sequence. The turn amino acid sequence may comprise 4 to 6 amino acid residues providing a stable turn structure; the turn sequence is flanked on one side by a first flanking sequence comprising SEQ ID NO: 1 and flanked on the other side by a second flanking sequence comprising SEQ ID NO: 2. The W residue at position 1 of SEQ ID NO: 1 may form a cross-strand indole-indole or cation-π interaction pair with the amino acid residue at position 3 of SEQ ID NO: 2 without any disulfide bond.

In the molecule as described above, the amino acid residue at position 2 of SEQ ID NO: 1 may be threonine; additionally, the amino acid residue at position 3 of SEQ ID NO: 1 may be tryptophan or tyrosine.

In the molecule as described herein, the amino acid residue at position 2 of SEQ ID NO: 2 may be threonine; additionally, the amino acid residue at position 3 of SEQ ID NO: 2 may be tryptophan or arginine.

In the molecule of the present invention, the collagen-binding linear hairpin peptide may comprise SEQ ID NO: 15, SEQ ID NO: 16, SEQ ID NO: 21, SEQ ID NO: 22, SEQ ID NO: 23, SEQ ID NO: 26, SEQ ID NO: 27, SEQ ID NO: 30, SEQ ID NO: 31, SEQ ID NO: 32, SEQ ID NO: 33, SEQ ID NO: 34, or SEQ ID NO: 35. Alternatively, the collagen-binding linear hairpin peptide may comprise SEQ ID NO: 17 SEQ ID NO: 18, SEQ ID NO: 19, SEQ ID NO: 20, SEQ ID NO: 24, SEQ ID NO: 25, SEQ ID NO: 28, SEQ ID NO: 29, SEQ ID NO: 36, SEQ ID NO: 37, SEQ ID NO: 38, SEQ ID NO: 39, SEQ ID NO: 40, or SEQ ID NO: 41.

In the molecule as described herein, Y may be KGG, acetyl, SEQ ID NO: 53, SEQ ID NO: 54, CGG, G, alginate-COOH, Dextran-COOH, or Dextran-NH2 or D-α-tocopheryl polyethylene glycol succinate (TPGS). Furthermore, in the molecule as described above, Z may be GGK, SEQ ID NO: 55, SEQ ID NO: 56, SEQ ID NO: 57, GGC, G, Fluor750, alginate-COOH, Dextran-COOH, Dextran-NH2, D-α-tocopheryl polyethylene glycol succinate (TPGS), SEQ ID NO: 58, SEQ ID NO: 59, SEQ ID NO: 60, SEQ ID NO:110, SEQ ID NO: 61, SEQ ID NO: 62 or SEQ ID NO: 63.

In specific embodiments of the present invention, the molecule of Formula (I) may comprise SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO:111, SEQ ID NO: 89, SEQ ID NO: 90, SEQ ID NO: 91, SEQ ID NO: 105 or SEQ ID NO:106.

The present invention further provides a method of delivering a compound of interest to a site of interest, the site of interest containing collagen. In the method, a molecule according to the present invention is provided at the site of interest; the collagen-binding linear hairpin peptide then binds to collagen at the site of interest, thereby delivering the compound of interest to the site of interest. The site of interest may be fibrotic or fibrosing tissue.

The present invention further provides a pharmaceutical composition comprising a molecule described herein and a pharmaceutically acceptable carrier, diluent or excipient.

Other aspects and features of the present disclosure will become apparent to those ordinarily skilled in the art upon review of the following description of specific embodiments in conjunction with the accompanying figures.

### BRIEF DESCRIPTION OF THE DRAWINGS

In order that the invention may be more clearly understood, embodiments thereof will now be described in detail by way of example, with reference to the accompanying drawings, in which:

FIG. 1: Defining the collagen-responsive region of a heat-sensitive thrombin inhibitor MH2-wZIP4 (SEQ ID NO:80) by use of two-dimensional (2D) NMR spectroscopy. FIG. 1A shows 2D H-¹⁵N HSQC spectra of ¹⁵N-labelled MH2-wZIP4 free (left) and after addition of collagen (right), showing differential binding to collagen. H-¹⁵N HSQC crosspeaks are labeled by their assignments to the sequence of MH2-wZIP4. FIG. 1B is a semi-quantitative comparison of the HSQC peak intensities for MH2-wZIP4 in the presence of collagen at two different experimental temperatures, 277 K and 298 K. HSQC peak intensities are scaled relative to those for MH2-wZIP4 free in solution. Filled bars are data of 277K; hatched bars are data of 298K; * indicates no HSQC peaks observed for the free peptide. The labels, ε8, ε10, ε17 and ε19, designate the side-chain aromatic H-¹⁵N HSQC crosspeaks of the four tryptophans, Trp8, Trp10, Trp17 and Trp19, respectively, in MH2-wZIP4. FIG. 1C is a quantitative evaluation of the impact of collagen using ¹⁵N-transverse (R₂) relaxation times. Filled bars are ¹⁵N-MH2-wZIP4; hatched bars are ¹⁵N-MH2-wZIP4 in collagen hydrogel. As in FIG. 1B, the labels, ε8, ε10, ε17 and ε19, designate the side-chain aromatic H-¹⁵N HSQC crosspeaks of the four tryptophans, Trp8, Trp10, Trp17 and Trp19, respectively. Data of FIGS. 1B and 1C were collected with a sample of ¹⁵N-labeled MH2-wZIP4 introduced through diffusion into a partially-aligned collagen hydrogel preformed under magnetic field guidance (Ni 2012). FIG. 1D shows 2D H-¹⁵N HSQC spectra of ¹⁵N-labelled MH2-wZIP4 mixed with collagen after removing collagen fibrils (polymers) and after degradation of collagen by a collagenase, demonstrating specificity of MH2-wZIP4 for unpolymerized collagen. I. MH2-wZIP4 in solution; II. MH2-wZIP4 in collagen hydrogel; III. MH2-wZIP4 in supernatant (after removal of polymerized collagen); IV. MH2-wZIP4 in supernatant (affer digestion of soluble collagen by a collagenase). The encircled H-¹⁵N HSQC crosspeaks in I and IV indicate those peaks that disappear in the presence of collagen (i.e. in II and III), but which re-appear after the destruction of collagen by a collagenase.

FIG. 2 shows the response to collagen binding of representative molecules containing linear hairpin peptides determined by use of proton NMR spectroscopy. (2A) proton NMR spectra of the bivalent thrombin inhibitor MH2-wZIP4 in the presence of collagen before (dotted lines) and after (solid lines) collagen gelation (polymerization) at 277K. (2B) proton NMR spectra of MH2-wZIP5/collagen before (dotted lines) and after (solid lines) collagen gelation at 277K. (2C) proton NMR spectra of MH2-wZIP6/collagen before (dotted lines) and after (solid lines) collagen gelation at 277K. (2D) proton NMR spectra of MH2-wZIP4/collagen solution before (dotted lines) and after (solid lines) the addition of a collagenase at 298K.

FIG. 3 shows the response to collagen of peptide fragments of the bivalent and heat-sensitive thrombin inhibitor MH2-wZIP4. FIG. 3A is a proton NMR spectrum of MH2-wZIP4-F1 (Table 4—SEQ ID NO: 68), free (dotted lines) in solution and after (solid lines) addition of collagen. (3B) proton NMR spectra of MH2-wZIP4-F2 (SEQ ID NO: 69). FIG. 3C is a proton NMR spectrum of MH2-wZIP4-F3 (Table 5—SEQ ID NO: 81), free (dotted lines) in solution and after (solid lines) addition of collagen. FIG. 3D is a proton NMR spectrum of MH2-wZIP4-F4 (Table 5—SEQ ID NO: 82) free (dotted lines) in solution and after (solid lines) addition of collagen.

FIG. 4 shows the response to collagen of other molecules containing trpzip hairpin peptides. FIG. 4A shows a proton NMR spectrum of an N-terminally extended derivative of MH2-wZIP4 (P4223; SEQ ID NO: 90, WO/2012/142696), free (dotted lines) in solution and after (solid lines) addition of collagen. FIG. 4B shows a proton NMR spectrum of MH2-wZIP1GG(SEQ ID NO: 79—from Ni 2012), free (dotted lines) in solution and after (solid lines) addition of collagen.

FIG. 5 shows the response of representative peptides to collagen as detected by proton NMR spectroscopy. FIG. 5A shows proton NMR spectra of gb1 (panel A), trpzip4 (panel B), trpzip5 (panel C) and trpzip6 (panel D) free (dotted lines) in solution and after (solid lines) addition of collagen (reproduced from Ni et al, PCT patent application, 2012). FIG. 5B shows proton NMR spectra of trpzip1 (panel A), v4-gp120 (panel B), v4-gp160 (Table 2; panel C) and HP7 (Table 4; panel D) free (dotted lines) in solution and after (solid lines) addition of collagen. FIG. 5C shows a proton NMR spectra of trpzip2 (panel A), trpzip7 (panel B), trpzip8 (panel C), and HP5W4 (Table 2; panel D) free (dotted lines) in solution and after (solid lines) addition of collagen. FIG. 5D shows proton NMR spectra of new hairpin-forming derivatives of the gb1 peptide (Table 2), e.g. CBLH1 (panel A), CBLH2 (panel B), CBLH3 panel C), CBLH4 (panel D) free (dotted lines) in solution and after (solid lines) addition of collagen. FIG. 5E shows proton NMR spectra of selected analogs of trpzip1 and trpzip2, i.e. Y4Y9-trpzip1 (panel A), Y4-trpzip1 (panel B), WYYW-trpzip2 (Table 2; panel C), and W2W11-trpzip2 (Table 4; panel D), free (dotted lines) in solution and after (solid lines) addition of collagen.

FIG. 6 shows two-dimensional H-¹⁵N HSQC spectra of a representative antibody VH domain protein modified at the N-terminus to include the collagen-binding trpzip4 segment. FIG. 6A is a H-¹⁵N HSQC spectrum of the free trpzip4-VH conjugate BRI-TP404, while FIG. 6B shows the same in the presence of collagen. FIG. 6C shows H-¹⁵N HSQC spectra of the BRI-TP404/collagen sample after collagen degradation by a collagenase.

FIG. 7 shows a two-dimensional H-¹⁵N HSQC spectrum of another representative antibody VH domain protein conjugated to the collagen-binding trpzip4 hairpin-IRFTD-trpzip4-GGS-PEPA1 or BRI-T404 (SEQ ID NO: 91, WO/2012/142696).

FIG. 8 shows retention of a representative molecule containing a hairpin peptide by collagen hydrogels shown by fluorescence imaging. FIG. 8A is a comparison of FL4247/collagen solutions loaded on to preformed collagen hydrogels (left, in form of disks at 100 μL each) with collagen hydrogels formed from pre-mixed 100 μl solutions of collagen and FL4247 (right). FIG. 8B monitors the diffusion of FL4247 out of collagen hydrogels formed from pre-mixed solutions of collagen and FL4247 (see FIG. 8A, right panel) into buffer solutions.

FIG. 9 shows the in vivo distributions of a fluorescently-labeled collagen-binding hairpin peptide FL4247 studied in mice and rat. FIG. 9A shows the fluorescence distribution following i.v. injection of 100 μl of saline-formulated FL4247 (dosage ˜0.25 mg/kg) in a normal mouse (body weight of ˜25 g). FIG. 9B shows the distribution following i.p. injection of 100 μl of saline-formulated FL4247 (dosage ˜0.25 mg/kg) in a normal mouse. FIG. 9C shows the clearance of FL4247 in a normal rat (body weight of ˜250 g) with injection of 100 μl of saline-formulated FL4247 (equivalent to a reduced dosage of 0.025 mg/kg).

FIG. 10 shows the local retention of two representative fluorescent molecules FL4247 and FL4447 containing two different CBLH peptides through in vivo imaging of a rat model of venous thrombosis. FIG. 10A are imaging data collected with FL4247 introduced via tail vein injection of 100 μL of stock FL4247 diluted in 700 μL of PBS, or at an approximate dosage of 0.2 mg/kg, 30 minutes after the injury of vena cava by 10% FeCl₃. The image in the left panel was taken immediately after probe injection and 30 minutes after FeCl₃ application, while the right panel image was taken 30 minutes after probe injection and 60 minutes after FeCl₃ application. FIG. 10B compares the fluorescence intensities of the opened vena cava and the separated thrombus collected through post-mortem extraction of the injured section of the vena cava. Values for the fluorescence intensities were obtained by quantifying individually the fluorescence images of the vena cava and the thrombus. The probe FL4312 is structurally identical to FL4247, but prepared from a different batch of synthesized P4247. FL4447 is a fluorescently-labeled CBLH peptide based on the sequence of CBLH5 (Table 5), while FL4434 is based on the non-functional gb1 peptide which does not bind collagen (Table 4). FL-Dye is the un-reacted dye collected from HPLC purifications of the fluorescently-labeled CBLH peptides. Dosage of injection for each fluorescent agent was approximately ¼^{th} of that used for FL4247 in FIG. 10A, i.e. ˜200 μL as compared to 800 μL in total injection volume.

FIG. 11 shows retention of a representative molecule FL4247 containing the collagen-binding trpzip4 hairpin shown by in vivo imaging of a mouse model of pulmonary fibrosis. FL4247 was introduced via tail vein injection (dosage ˜0.5 mg/kg or 200 μl of saline-formulated FL4247). The image in the left panel was taken immediately following injection, while the right panel image was taken 22 minutes after injection.

### DESCRIPTION OF PREFERRED EMBODIMENTS

The present invention relates to derivatives of collagen-binding hairpin peptides, particularly peptides for imaging and drug delivery to fibrosing/fibrotic tissues.

In one aspect of the invention there is provided a molecule of Formula (I),

(Y)ₙ-(CBLH)-(Z)ₘ (I)

Y is a first compound of interest and Z is a second compound of interest, where Y and Z may be the same or different; n and m are independently 0 or 1 with the proviso that at least one of n and m is 1. CBLH is a collagen-binding linear hairpin peptide.
The collagen-binding linear hairpin (CBLH) peptide in the molecule described herein binds to collagen, particularly to type-I collagen, the most abundant extracellular protein in living tissues; type-I collagen is also over-accumulated in fibrotic organs and in inflamed vascular lesions. More specifically, the CBLH peptides have affinity only for unpolymerized or monomeric collagen abundant in fibrotic/fibrosing tissues undergoing active and uncontrolled remodelling. Therefore, the CBLH peptides lack affinity for well-aligned collagen fibrils in healthy tissues, binding only fibrotic/fibrosing tissues, thereby conferring utility for targeting fibrotic tissues and for imaging and localized drug delivery.

The CBLH peptide may comprise 19 or fewer amino acids and a turn amino acid sequence comprising 4 to 6 amino acid residues providing a stable turn structure. The turn sequence may be flanked on one side by a first flanking sequence comprising SEQ ID NO: 1 and on the other side by a second flanking sequence comprising SEQ ID NO: 2. The W residue at position 1 of SEQ ID NO: 1 forms a cross-strand indole-indole or cation-π interaction pair with the amino acid residue at position 3 of SEQ ID NO: 2 without any disulfide bond. The collagen-binding linear hairpin peptide binds to monomeric or unpolymerized collagen at the site of interest thereby delivering the compound of interest to the site of interest.

The collagen-binding linear hairpin (CBLH) peptide comprises less than 20 amino acid residues that possess an autonomously stable three-dimensional structure free from disulfide bonds. The turn amino acid sequence is any amino acid sequence that comprises 4 to 6 amino acid residues and provides a stable turn structure. Some examples of suitable turn sequences are DDATKT (SEQ ID NO: 3), EpNK (SEQ ID NO: 4), ENGK (SEQ ID NO: 5), EGNK (SEQ ID NO: 6), NGSA (SEQ ID NO: 7), NGTN (SEQ ID NO: 8), NGSTA (SEQ ID NO: 9), NDSN (SEQ ID NO: 10), NNSA (SEQ ID NO: 11), NNST (SEQ ID NO: 12), NGSN (SEQ ID NO: 13) and NPATGK (SEQ ID NO: 14).

The first flanking sequence of the CBLH peptide is SEQ ID NO: 1, which is WX₁X₂ where: X₁ is T, R, H, V, I, L, N, K, A, F, Y or W; and, X₂ is W, Y, F or K. The second flanking sequence of the CBLH peptide is SEQ ID NO: 2, which is X₁X₂ X₃ where: X₁ is W, Y, F or K; X₂ is T, R, H, V, I, L, N, K, A, F, Y or W; and, X₃ is W or R. The amino acid residue at position 1 of SEQ ID NO: 1 is tryptophan. The amino acid residue at position 2 of SEQ ID NO: 1 is preferably threonine. The amino acid residue at position 3 of SEQ ID NO: 1 is preferably tryptophan or tyrosine. The amino acid residue at position 1 of SEQ ID NO: 2 is preferably tryptophan. The amino acid residue at position 2 of SEQ ID NO: 2 is preferably threonine. The amino acid residue at position 3 of SEQ ID NO: 2 is tryptophan or arginine. Thus, the first flanking sequence preferably has a WT(W/Y) motif. The second flanking sequence preferably has a WT(W/R) motif. When the amino acid residue at position 3 of SEQ ID NO: 2 is tryptophan, the tryptophan residue at position 1 of SEQ ID NO: 1 forms a cross-strand indole-indole bond with the tryptophan at position 3 of SEQ ID NO: 2. When the amino acid residue at position 3 of SEQ ID NO: 2 is arginine, the tryptophan residue at position 1 of SEQ ID NO: 1 forms a cross-strand cation-π interaction pair with the arginine at position 3 of SEQ ID NO: 2.

Some representative examples of collagen-binding linear hairpin (CBLH) peptides are provided in Table 2.

TABLE 2

| Name | Source | Sequence |
|---|---|---|
| trpzip6(**) | Cochran 2001b | GEWTWDDATKTWTVTE (PS + NMR) |
| | | SEQ ID NO: 15 |
| trpzip5(**) | Cochran 2001b | GEWTYDDATKTFTWTE ( PS + NMR) |
| | | SEQ ID NO: 16 |
| trpzip4(**) | Cochran 2001b | GEWTWDDATKTWTWTE (PS + NMR) |
| | | SEQ ID NO: 17 |
| trpzip3 | Cochran 2001b | SWTWEpNKWTWK |
| | | SEQ ID NO: 18 |
| trpzip2(**) | Cochran 2001b | SWTWENGKWTWK (PS + NMR) |
| | | SEQ ID NO: 19 |
| trpzip1(**) | Cochran 2001b | SWTWEGNKWTWK (PS + NMR) |
| | | SEQ ID NO: 20 |
| Y4-trpzip1 | Takekiyo 2009 | SWTYEGNKWTWK (PS + NMR) |
| | | SEQ ID NO: 21 |
| Y4Y9-trpzip1 | Takekiyo 2009 | SWTYEGNKYTWK (PS + NMR) |
| | | SEQ ID NO: 22 |
| WYYW-trpzip2 | Wu 2009 | SWTYENGKYTWK (PS + NMR) |
| | | SEQ ID NO: 23 |
| v4-gp120 | Cochran 2001b | TWTWNGSAWTWN (PS + NMR) |
| (Q77430-9HIV1)(**) | UNIPROTKB | SEQ ID NO: 24 |
| v4-gp120-ext | UNIPROTKB | STWTWNGSAWTWNE |
| (Q77430-9HIV1) | | SEQ ID NO: 25 |
| v4-gp160 | UNIPROTKB | TWTWNGTNWTRN (PS + NMR) |
| (Q6UYR0-9H1V1)(**) | | SEQ ID NO: 26 |
| v4-gp160-ext(**) | UNIPROTKB | STWTWNGTNWTRND (PS + NMR) |
| (or CBLH5) | | SEQ ID NO: 27 |
| (Q6UYR0-9H1V1) | | |
| pp-D7EUT8 | UNIPROTKB | TWRWNGSTAWTWS (PS + NMR) |
| (D7EUT8-MYCTU) | | SEQ ID NO: 28 |
| pp-D7EUT8-ext | UNIPROTKB | STWRWNGSTAWTWSTAS |
| (D7EUT8-MYCTU) | | SEQ ID NO: 29 |
| v4-gp120v2 | Zhu 1993 | TWTRNDSNWTWN |
| | | SEQ ID NO: 30 |
| v4-gp120v2-ext | Zhu 1993 | STWTRNDSNWTWNG |
| | | SEQ ID NO: 31 |
| pp-Q77413 | UNIPROTKB | TWTRNNSAWTWN |
| (Q77413-9HIV1) | | SEQ ID NO: 32 |
| pp-Q77413-ext | UNIPROTKB | STWTRNNSAWTWNG |
| (Q77413-9HIV1) | | SEQ ID NO: 33 |
| pp-Q77425 | UNIPROTKB | TWTRNNSTWTWN |
| (Q77425-9HIV1) | | SEQ ID NO: 34 |
| pp-Q77425-ext | UNIPROTKB | STWTRNNSTWTWNG |
| (Q77425-9HIV1) | | SEQ ID NO: 35 |
| pp-Q904S5 | UNIPROTKB | TWAWNGSNWTWN |
| (Q904S5-9HIV1) | | SEQ ID NO: 36 |
| pp-Q904S5-ext | UNIPROTKB | STWAWNGSNWTWNG |
| (Q904S5-9HIV1) | | SEQ ID NO: 37 |
| trpzip7(**) | Russel 2003 | GEWHWDDATKTWVWTE (PS + NMR) |
| | | SEQ ID NO: 38 |
| trpzip8(**) | Russel 2003 | GEWVWDDATKTWHWTE (PS + NMR) |
| | | SEQ ID NO: 39 |
| trpzip9 | Russel 2003 | GEWVWDDATKTWVWTE |
| | | SEQ ID NO: 40 |
| HP5W4(**) | Fesinmeyer 2004 | KKWTWNPATGKWTWQE (PS + NMR) |
| | | SEQ ID NO: 41 |
| W12W14-gb1 | presently | GEWTYDDATKTWTWTE (PS + NMR) |
| | described | SEQ ID NO: 97 |
| W5W12R14-gb1 | presently | GEWTWDDATKTWTRTE (PS + NMR) |
| | described | SEQ ID NO: 98 |
| W12W14-gb1-AL | presently | GEWTYNPATGKWTWTE (PS + NMR) |
| (or CBLH1) | described | SEQ ID NO: 99 |
| W5W12R14-gb1-AL | presently | GEWTWNPATGKWTRTE (PS + NMR) |
| (or CBLH2) | described | SEQ ID NO: 100 |
| K1W12W14-gb1 | presently | KEWTYDDATKTWTWTE (PS + NMR) |
| | described | SEQ ID NO: 101 |
| K1W5W12R14-gb1 | presently | KEWTWDDATKTWTRTE (PS + NMR) |
| | described | SEQ ID NO: 102 |
| K1W12W14-gb1-AL | presently | KEWTYNPATGKWTWTE (PS + NMR) |
| (or CBLH3) | described | SEQ ID NO: 103 |
| K1W5W12R14-gb1-AL | presently | KEWTWNPATGKWTRTE (PS + NMR) |
| (or CBLH4) | described | SEQ ID NO: 104 |

(**)indicates that these peptides have been produced via the respective routes of synthesis (PS) and that their collagen-binding properties have been demonstrated by use of NMR spectroscopy (NMR). All the peptides here were synthesized as C-terminal amides in accordance with the design of the trpzip peptides based on the gb1 sequence (Cochran 2001b). This includes HP5W4 which was designed originally as a C-terminal carboxylate (Fesinmeger 2004).
(***)UNIPROTKB is an online software tool offering “Blast”-based search and comparison with protein sequences in genomic databases.

Molecules useful in the present invention comprise at least one compound of interest (Y and/or Z). In some embodiments, where both n and m are 1, the molecule comprises two compounds of interest, i.e. both Y and Z are present. The compounds of interest (Y and Z) may be the same or different. The compounds of interest are covalently linked to the collagen-binding linear hairpin (CBLH) peptide through chemical conjugation. Compounds of interest may be any compound that can be conjugated to a CBLH peptide using standard linkers and conjugation chemistry. Such compounds may be, for example, active pharmaceutical ingredients (APIs), probes or inactive carriers. Particular examples of compounds of interest include imaging probes (e.g. fluorescent probes, magnetic resonance probes, surface plasmon resonance probes), small-molecule drugs, small peptides (e.g. thrombin binding peptides, pharmacologically active peptides), proteins (e.g. antigens, antibodies), polysaccharides, and/or nanoparticles (e.g. polymeric or metallic). Some representative examples of linkers and compounds of interest (i.e. Y and Z moieties) are provided in Table 3.

TABLE 3
Non-limiting examples of linkers and compounds of interest (Y and Z moieties).
It should be noted that the listed compounds of interest may be used interchangeably as
Y or Z moieties, and may be combined.

| Name | Source | Y moiety | Z moiety |
|---|---|---|---|
| GGK linker | presently | | GGK |
| | described | | |
| KGG linker | presently | KGG | |
| | described | | |
| N-terminal cap | presently | acetyl | |
| | described | | |
| Thrombin inhibitor | WO/2012/142696 | IRFTD | |
| | | (SEQ ID NO: | |
| | | 53) | |
| Thrombin inhibitor | WO/2012/142696 | FQPRPRFTD | |
| | | (SEQ ID NO: | |
| | | 54) | |
| CGG linker | presently | CGG | |
| | described | | |
| Thrombin inhibitor | WO/2012/142696 | | GDFEEIPEEYL |
| | | | (SEQ ID NO: 55) |
| Thrombin inhibitor | WO/2012/142696 | | GDFEEIPEEYLQ |
| | | | (SEQ ID NO: 56) |
| Thrombin substrate | presently | | GSFNPRGS |
| | described | | (SEQ ID NO: 57) |
| GGC linker | presently | | GGC |
| | described | | |
| G spacer | presently | G | G |
| | described | | |
| Thiol-reactive probe | AnaSpec, CF | | HiLyte Fluor750 |
| polymer carrier | | alginate- | alginate-COOH |
| | | COOH | |
| polymer carrier | | Dextran-COOH | Dextran-COOH |
| polymer carrier | | Dextran-NH2 | Dextran-NH2 |
| Micelle-forming lipid | | D-α-tocopheryl | D-α-tocopheryl |
| | | polyethylene | polyethylene glycol |
| | | glycol succinate | succinate (TPGS) |
| | | (TPGS) | |
| GGS-VHpro10 | presently | | GGS-VHpro10 |
| | described | | (SEQ ID NO: 58) |
| GDFEE-VHpro10 | presently | | GDFEE-VHpro10 |
| | described | | (SEQ ID NO: 59) |
| GGGGSS-VHpro10 | presently | | GGGGSS-VHpro10 |
| | described | | (SEQ ID NO: 60) |
| GGGSS-VHpro10 | presently | | GGGSS-VHpro10 |
| | described | | (SEQ ID NO: 110) |
| GGS-VHpro5 | presently | | GGS-VHpro5 |
| | described | | (SEQ ID NO: 61) |
| GGS-VHpro10c | presently | | GGS-VHpro10c |
| | described | | (SEQ ID NO: 62) |
| GGS-VHpro5c | presently | | GGS-VHpro5c |
| | described | | (SEQ ID NO: 63) |

In Table 3, VHpro10, VHpro5, VHpro10c and VHpro5c are examples of antibody V_{H}H domains that are ligands specific for human prothrombin. The amino acid sequences of these are as follows:

VHpro10
(SEQ ID NO: 64)
DVQLQASGGGLVQAGGSLRLTCAASGRTFDRYGWFRQAPGKEREFVASIG
TRLHYADSVKGRFTISRDNAKSTAFLEMNSLKPEDTAVYYCAAAESTRNW
YYKMSNDYDYWGQGTQVTVSSLEHHHHHH
VHpro5
(SEQ ID NO: 65)
DVQLQASGGGLVQAGGSLRLTCAASGRTFSSLSIAWFRQAPGKEREFVAG
IRWTAGSKTYANWVKGRFTISRDNAKSTAFLEMNSLKPEDTAVYYCAADN
ISDWGISKQLRTYHYWGQGTQVTVSSLEHHHHHH
VHpro10c
(SEQ ID NO: 66)
CHNDGGGGSDVQLQASGGGLVQAGGSLRLTCAASGRTFDRYGWFRQAPGK
EREFVASIGTRLHYADSVKGRFTISRDNAKSTAFLEMNSLKPEDTAVYYC
AAAESTRNWYYKMSNDYDYWGQGTQVTVSSLEHHHHHH
VHpro5c
(SEQ ID NO: 67)
CHNDGGGGSDVQLQASGGGLVQAGGSLRLTCAASGRTFSSLSIAWFRQAP
GKEREFVAGIRWTAGSKTYANWVKGRFTISRDNAKSTAFLEMNSLKPEDT
AVYYCAADNISDWGISKQLRTYHYWGQGTQVTVSSLEHHHHHH

In the molecule described herein, the compound(s) of interest may be linked to the CBLH peptide using a linker; such a linker should be of sufficient length and appropriate composition to provide flexible attachment of the molecule(s), but should not hamper the collagen-binding properties of the molecule. For example, and without wishing to be limiting, the linkermay be selected from G, GGK, GGC, CGG, KGG, GGS, GGGSS (SEQ ID NO:107), GGGGSS (SEQ ID NO:108), and GDFEE (SEQ ID NO:109).

The compound(s) of interest in the molecule of the present invention may also comprise additional sequences to aid in expression, detection or purification of a recombinant antibody or fragment thereof. Any such sequences or tags known to those of skill in the art may be used. For example, and without wishing to be limiting, the antibody or fragment thereof may comprise a targeting or signal sequence (for example, but not limited to ompA), a detection tag (for example, but not limited to c-Myc), a purification tag (for example, but not limited to a His₅ or His₆), or a combination thereof. In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

In yet another aspect of the present invention, there is provided a pharmaceutical compositions comprising a molecule of the present invention and a pharmaceutically acceptable carrier, diluent or excipient.

Pharmaceutical compositions comprise a molecule of the present invention and a pharmaceutically acceptable carrier, diluent or excipient. In one embodiment, a collagen-binding hairpin peptide may be covalently coupled to a carrier, diluent or excipient, in which case one of the compounds of interest in the molecule would be the carrier, diluent or excipient. In another embodiment, the pharmaceutical composition is a vaccine in which collagen-binding hairpin peptide is covalently conjugated to an antigen or other immune-response stimulating agent.

Pharmaceutical compositions may be formulated in a dosage form. Dosage forms include powders, tablets, capsules, softgels, solutions, suspensions, emulsions and other forms that are readily appreciated by one skilled in the art. The compositions may be administered orally, parenterally, intravenously or by any other convenient method. Some pharmaceutically acceptable carriers, diluents or excipients include, for example, antiadherents, binders (e.g. starches, sugars, cellulose, hydroxypropyl cellulose, ethyl cellulose, lactose, xylitol, sorbitol and maltitol), coatings (e.g. cellulose, synthetic polymers, corn protein zein and other polysaccharides), disintegrants (e.g. starch, cellulose, cross-linked polyvinyl pyrrolidone, sodium starch glycolate and sodium carboxymethyl cellulose), fillers/diluents (e.g. water, plant cellulose, dibasic calcium phosphate, vegetable fats and oils, lactose, sucrose, glucose, mannitol, sorbitol and calcium carbonate), flavors and colors, glidants, lubricants (e.g. talc, silica, vegetable stearin, magnesium stearate and stearic acid), preservatives (e.g. vitamin A, vitamin E, vitamin C, selenium, cysteine, methionine, citric acid, sodium citrate, methyl paraben and propyl paraben), antioxidants, sorbents, sweeteners, and mixtures thereof. Molecules or compositions of the present invention are packaged in a commercial package together with instructions for their use. Such packages are known to one skilled in the art and include, for example, bottles, jars, blister packs, boxes, etc.

Molecules and compositions of the present invention are particularly useful in medical applications for diagnosis and treatment of diseases and other conditions in a subject. The subjects may be human or other animals, especially those with blood circulatory systems, particularly mammals, for example, humans, dogs, cats, horses and rodents (e.g. hamsters, mice and rats). Because the CBLH peptides bind to collagen, the invention is particularly effective for delivering a compound of interest to fibrotic or fibrosing tissue.

Some exemplary applications of the method and molecules of the present invention include the following. Fluorescently-labelled CBLH peptides or those conjugated by other imaging probes can localize in fibrotic/fibrosing tissues for diagnosis of diseases involving fibrosis. CBLH peptides can be developed as universal tags for recombinant production of polypeptides and proteins with specificity to inflamed/fibrosing tissues. Conjugates of nanoparticulate carriers or polymers with CBLH peptides can enable localized delivery of pharmaceutical payloads or compositions to disease-specific tissues. CBLH peptides can also be conjugated directly to peptide-based or polysaccharide antigens and small-molecule drugs to achieve tissue-specific accumulation of vaccines and/or release of active drugs for treating a large number of pathologies and diseases, such as infections, atherosclerosis, cancer, arthritis etc., all with aberrant tissue (ECM) remodeling and fibrosis (Wynn 2004; Caravan 2007; Muzzard 2009).

Molecules of the present invention comprising collagen-binding hairpin peptides are of particular utility for imaging and drug delivery to inflamed mucosal surfaces in the nasal, oral and gastrointestinal (GI) cavities or tracks. For example, conventional strategies of immunization through systemic administration have been found to be sub-optimal for the control of bacterial colonization in the oral cavity, such as dental caries (Koga 2002; Chen 2010). The effectiveness of anti-microbial agents therefore require the development of dentotropic (localized) delivery systems, i.e. targeting agents specific to the dental structures, especially the dentin (Chen 2010). Tissue inflammation on mucosal surfaces, such as in the oral and nasal cavities, is known to lead to local accumulation of collagen (Liu 1990; Switalski 1993; Love 1997; Sciotti 1997; Petersen 2001; Koga 2002; Rivas 2004; Nikawa 2006). Molecules of the present invention (i.e. conjugates of collagen-binding hairpin peptides) are therefore expected to render therapeutic agents or vaccines particularly efficacious when the conjugates are delivered through oral or intranasal administration.

In a particularly interesting application adapted from previous work (Abou Neel 2012), therapeutic collagen preparations based on molecules of the present invention may be prepared whereby unpolymerized or monomeric collagen is complexed or blended with one or more active pharmaceutical ingredients conjugated with collagen-binding hairpin peptides to further increase local delivery to a site of interest, especially into fibrotic/fibrosing tissues. Drug carriers are commonly utilized to improve the bioavailability of orally-delivered drugs, specifically through molecular adhesion to the epithelial surfaces of the body (Peppas 2009). Non-covalent blends of collagen with polymer carriers and/or active pharmaceutical ingredients conjugated with collagen-binding hairpin peptides therefore have the potential to increase the retention of drugs by epithelial tissues undergoing inflammation and active fibrosis.

Thus, the present invention further provides a method of delivering a compound of interest to a site of interest, the site of interest containing collagen. The method may comprise providing a molecule as described herein at the site of interest. The collagen-binding linear hairpin peptide binds to collagen at the site of interest, thereby delivering the compound of interest to the site of interest.

The present invention will be further illustrated in the following examples. However, it is to be understood that these examples are for illustrative purposes only and should not be used to limit the scope of the present invention in any manner.

### Example 1

### Defining the Sequence Characteristics of Collagen-Binding Hairpin Peptides

Table 2 shows a list of representative peptides that are selected as collagen binders based on their sequence characteristics. Reported collagen binders (Table 1) share two important structural features: (1) almost all (except for one) contain at least one Trp residue and (2) peptide cyclization (mostly via a Cys/Cys disulfide bond) was employed to reduce the conformational flexibility and potentially to increase binding affinity to collagen. We hypothesize that well-structured peptides abundant in Trp residues may also have the capacity to bind to collagen. In this regard, indole-indole interactions between two strategically-located Trp residues have been shown to stabilize the native hairpin structure of a bioactive peptide in place of a covalent disulfide bond between a pair of Cys residues (Mirassou 2009). This invention therefore focuses on well-structured linear hairpin peptides particularly rich in Trp residues (Santiveri 2010) as ligands of collagen (Table 2), in particular of unpolymerized soluble collagen.

As demonstrated in our previous patent application (WO/2012/142696), four (4) trpzip peptides, i.e. trpzip1, trpzip4, trpzip5 and trpzip6 (Table 2), all responded to collagen in terms of differential perturbations including resonance broadening of the peptide proton NMR spectra in the presence of collagen (see Example 4). On the other hand, the gb1 peptide (Table 4) containing the parent sequence of trpzip4, trpzip5 and trpzip6 (Cochran 2001b) showed no response to collagen under a variety of experimental conditions. The best-structured linear hairpin peptide, trpzip4, showed the most pronounced changes in response to the presence of collagen, indicating that two trpzip motifs, i.e. WTW, located on both strands of the hairpin structure may confer specific binding to collagen (see Example 4) in addition to being the structure-stabilizing force for the linear peptide (Cochran 2001b). Three selection criteria are utilized to define the structural characteristics of collagen-binding (responsive) peptides: (1) being linear; (2) having at least two Trp residues; (3) having a significantly folded hairpin structure. Representative peptides satisfying these three criteria are listed in Table 2. All peptides have a WT₁W₁ motif in the N-terminal region followed by a turn (loop) sequence and in the C-terminal region a second W₂T₂(W/R) motif potentially stabilizing a well-structured hairpin fold. Therefore, two WTW motifs are found in trpzip1, trpzip2, trpzip3, trpzip4, HP5W4 and in the V4 loop peptide of the HIV envelop protein gp120 (Q77430-9H1V1) (Table 2). Trpzip6 has the WTW motif on the first strand of a putative hairpin structure, while the second strand has the KTW variant of the W₂T₂(W/R) motif. The trpzip5 peptide has the WTY motif in the N-terminal region and a FTW variant in the C-terminal strand. In trpzip7, trpzip8 and trpzip9, the WTW motifs are substituted by WVW and WHW, respectively. In hairpin sequences of gp120 variants (Table 2), some WTW motifs are substituted by WTR, which may provide stabilization for the hairpin structure through strong cation-π interactions (Santiveri 2010; Chen 2005). A putative protein predicted from the genome sequence of the infectious agent Mycobacterium tuberculosis also contains a hairpin-like sequence rich in Trp, TWRWNGSTAWTWS (Table 2) with the WRW sequence as the first WT₁W₁ motif. It is important to note that a single WLW motif is already found in a collagen-binding ligand mimicking the vWF protein (Depraetere 1998), i.e. in two peptides called the Q-peptide and the N-peptide (Table 1).

### Example 2

### Collagen-Binding Properties of Polypeptides Containing Hairpin Motifs

Rat collagen hydrogels were prepared using rat-tail collagen type I (at 4 mg/ml in 0.02 N acetic acid) from BD Bioscience. A volume of 200 μL of the concentrated collagen solution was mixed with an equal volume of a buffer solution that was 400 mM in Tris-HCl and 400 mM in NaCl with a pH of 7.6 and with 50 μL of deuterated water (D₂O). The sample mixture was transferred to an NMR tube followed by gentle mixing under agitation using a Thermolyne™ Max Mix-II apparatus. The NMR tube containing the collagen solution was placed within the RF probe housed in a 500 MHz super-conducting magnet (Bruker Avance-500 NMR spectrometer). The probe and sample bulk temperature was kept at 277 K for 3 hours and then elevated to 310 K at a rate of 1 degree/10 min to enable fibril alignment during a slow process of collagen gelation under the influence of the magnetic field (Ma 2008). The degree of fibril alignment of the collagen hydrogels was determined by use of deuterium NMR spectroscopy of the added D₂O as described (Ma 2008).

Human collagen hydrogels were prepared using either human placenta collagen type I from BD Bioscience (at 2.23 mg/ml in 2 mM HCl) or the VitroCol™ preparation of human collagen from Advanced BioMatrix (at 2.9 mg/ml in 0.01 N HCl). A volume of 400 μL of the concentrated collagen solution was mixed with 50 μL of the solution of 10×PBS (phosphate-buffered saline) supplemented by Na₂PO₄ at 500 mM and pH 7.4 and with 50 μL of deuterated water (D₂O). The sample mixture was transferred to an NMR tube followed by gentle mixing before being subjected to the same gelation process as described above.

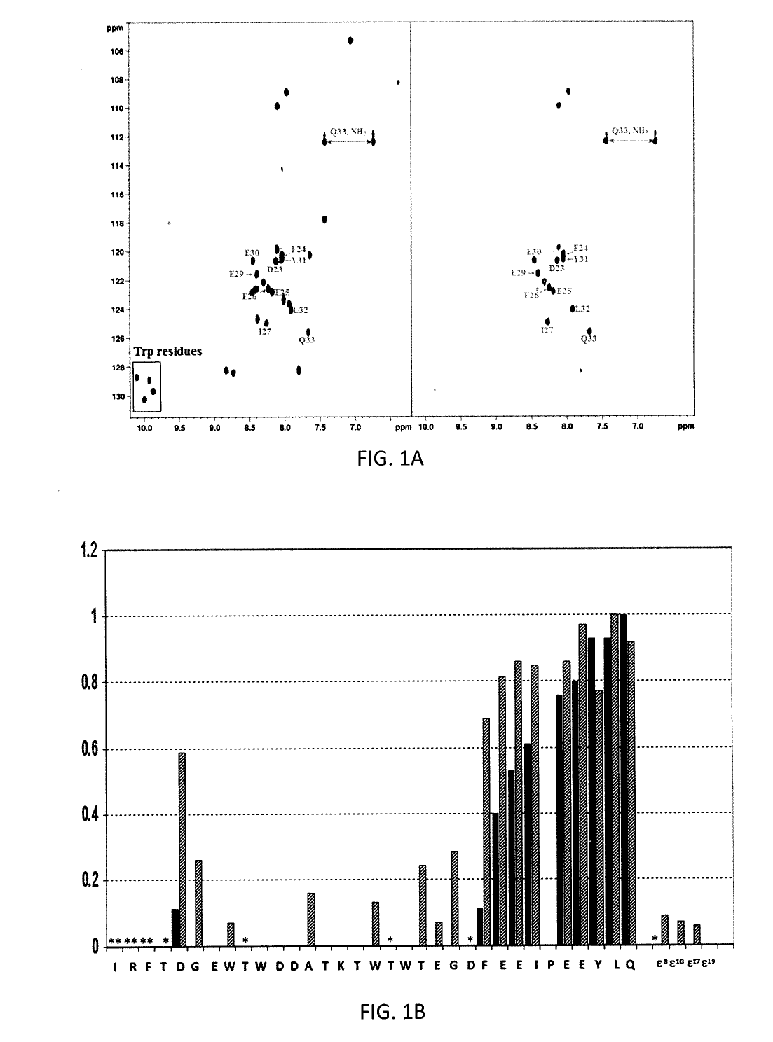

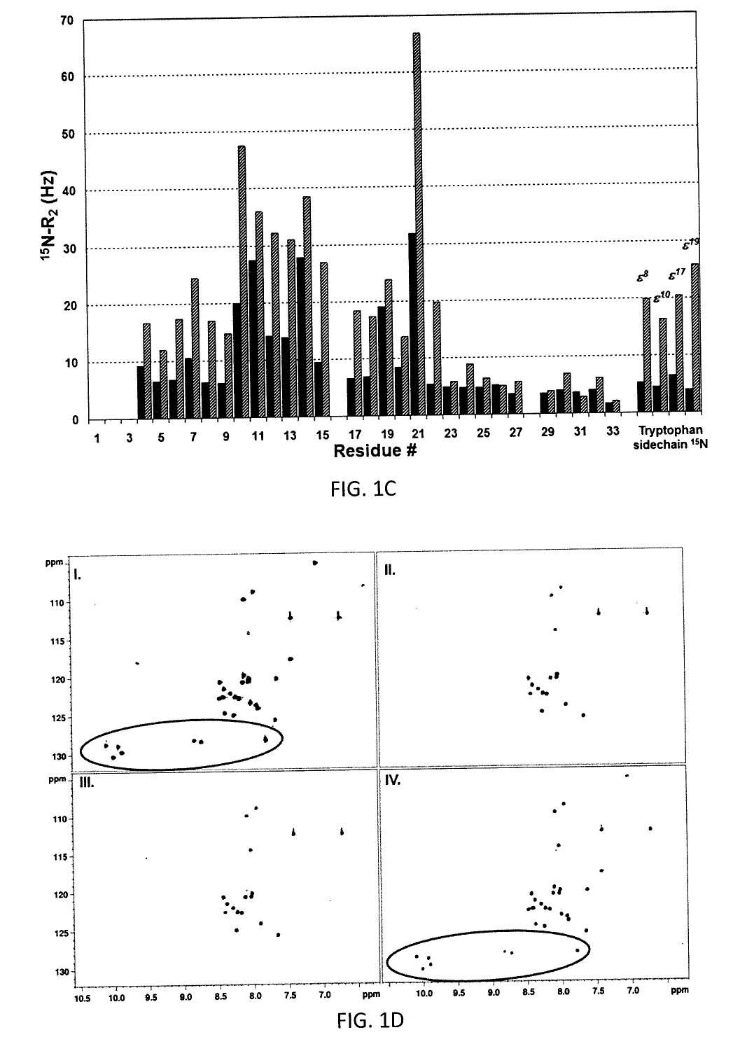

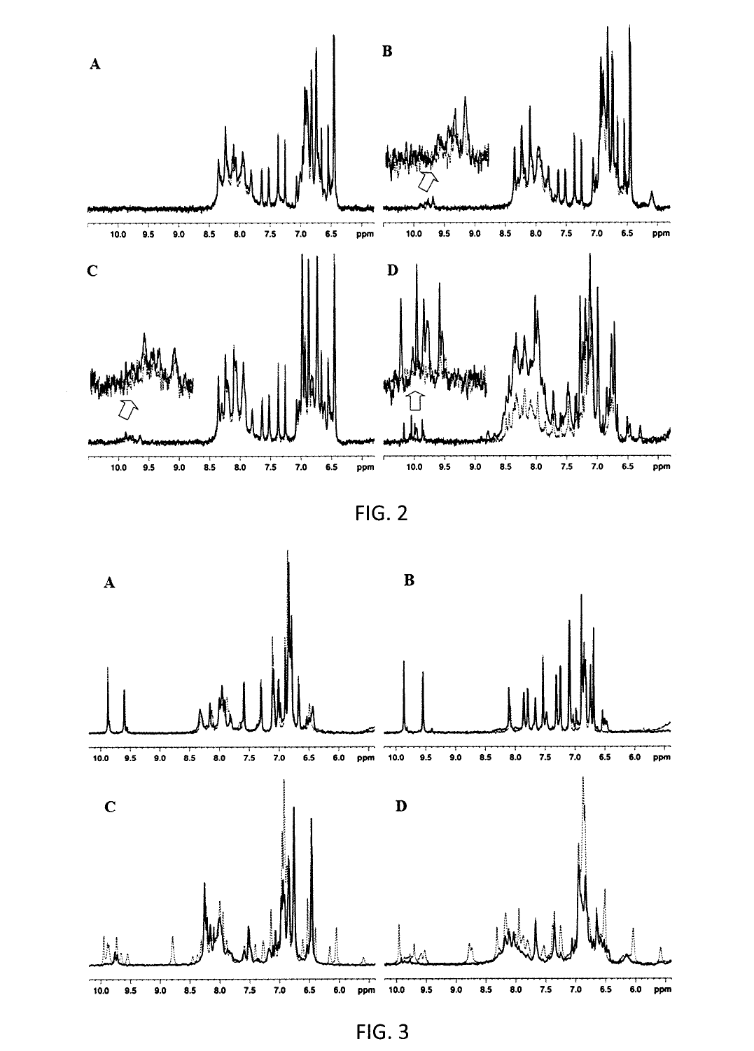

Randomly-deposited collagen (hydrogel) matrix was prepared following the same procedure as above for partially aligned hydrogels, except that the RF probe and the NMR tube (or test tubes) containing the collagen solution were placed outside the magnetic field. Peptides containing collagen-binding hairpins were introduced into the hydrogels in two ways, the first with the collagen stock solution diluted (50:50 in volume ratio) by the buffer of 400 mM in Tris-HCl and 400 mM in NaCl at pH 7.6 containing the peptides of interest (i.e. for FIGS. 1A & 1D), which gelates in the presence of the added peptide. Alternatively, peptides in an appropriate buffer were introduced to the top of the hydrogel matrix (i.e. for FIGS. 1B & 1C) preformed in the NMR tube (or test tube) following the procedures described above. The behavior of the peptides in the hydrogels was followed by use of NMR H-¹⁵N HSQC spectra for ¹⁵N-labelled peptides (FIG. 1) and/or by use of one-dimensional proton NMR spectroscopy (FIG. 2).

The autonomous or modular nature of collagen-binding linear hairpin (CBLH) peptides was demonstrated by the NMR data for thrombin inhibitors harboring the sequences of these CBLH peptide, in particular MH2-wZIP4, MH2-wZIP5 and MH2-wZIP6 (WO/2012/142696). MH2-wZIP4 exhibited significantly altered properties and/or a differential entrapment in the presence of collagen or collagen hydrogels (FIG. 1A, 1B and 1C). Most importantly, ¹⁵N-MH2-wZIP4 in the collagen hydrogel is shown to have the sequence moiety GD₂₃FEEIP₂₈EEYLQ₃₃ fully exposed and freely available (FIG. 1A, right panel and FIG. 1B). Proton NMR spectra also demonstrated that both MH2-wZIP5 (FIG. 2B) and MH2-wZIP6 (FIG. 2C) exhibit varying degrees of entrapment in the collagen (gel) matrix. In general, MH2-wZIP4 experiences the most pronounced proton NMR spectral changes within a hydrogel formed from collagen premixed with the peptide (FIG. 2). The pronounced changes of MH2-wZIP4 are followed by MH2-wZIP6 and by MH2-wZIP5 in their differential resonance perturbations within the collagen hydrogel. Such pronounced degrees of proton NMR line broadening, i.e. MH2-wZIP4>MH2-wZIP6>MH2-wZIP5 are always accompanied by reduced fibril alignment of the hydrogels formed from the corresponding peptide-collagen complex, as determined by use of deuterium NMR spectroscopy (see below for further details).

Deuterium NMR spectroscopy studies of collagen responsiveness (and/or binding) for each peptide were carried out using a volume of (400-X) μL of a peptide dissolved in pure water mixed with 50 μL of a 10×PBS (phosphate-buffered saline) supplemented by sodium phosphate to a final concentration of 50 mM, with 50 μL of deuterated water (D₂O) to form the reference sample (where X=0), or the peptide-collagen complex with X μL of rat-tail collagen type I (at 3.6 to 4 mg/ml in 0.02 N acetic acid from BD Bioscience) adjusted to achieve the desired final concentration of collagen. The pH value of the reference peptide samples was adjusted to match the pH of the final peptide-collagen complex in order to facilitate NMR spectral comparison. The peptide-collagen solutions were also subjected to gelation under magnetic field guidance followed by the measurement of fibril alignment (Ma 2008) as a semi-quantitative indication of peptide-collagen binding. Deuterium coupling constants measuring the degree of collagen fibril alignment were reduced by more than 1.0 Hz for MH2-wZIP4, by less than 0.5 Hz for MH2-wZIP6 and MH2-wZIP5, which parallel those observed for trpzip4, trpzip6, trpzip5 and the gb1 peptide (see Example 4). As a comparison, a solution of rat collagen at a concentration of 1.8 mg/ml in 200 mM Tris-Cl/200 mM NaCl at pH 7.6 formed a partially-aligned hydrogel with a deuterium coupling constant (Ma, 2008) of 2.5 Hz, while a solution of rat collagen at a concentration of 3 mg/ml in PBS (137 mM NaCl/2.68 mM KCl/10.1 mM Na₂HPO₄/1.76 mM KH₂PO₄) supplemented with Na₂HPO₄ to achieve a final concentration of 50 mM at pH 7.4 formed a partially-aligned hydrogel with a deuterium coupling constant of 4.8 Hz. A solution of human collagen at a concentration of 2.23 mg/ml in PBS supplemented with Na₂HPO₄ with a final concentration of 50 mM at pH 7.4 formed a partially-aligned hydrogel with a deuterium coupling constant of 1.3 Hz.

The MH2-wZIP4 molecule exhibited a greatly-reduced entrapment when diffused into preparations of collagen hydrogels that are pre-aligned to mimic the collagen matrix in healthy tissues (WO/2012/142696). Looking more closely at FIG. 1C, the ¹⁵N-NMR transverse relaxation rates (R₂) of ¹⁵N-MH2-wZIP4 show, quantitatively, the differential behavior of each residue in response to the presence of partially-aligned collagen. Therefore, residues in the entire region of G₂₂DFEEI₂₇PEEYL₃₂Q in MH2-wZIP4 had the least changes in their ¹⁵N—R₂ values when comparing ¹⁵N-MH2-wZIP4 in the control (buffer) solution and in partially-aligned collagen hydrogels. Such low ¹⁵N—R₂ values for these residues signify the lack of perturbations on these residues by the presence of the collagen matrix, a property of MH2-wZIP4 already evident at the level of (H,¹⁵N)-HSQC spectral intensities (FIG. 1B). In contrast, the trpzip4 moiety of MH2-wZIP4 exhibit significantly increased ¹⁵N—R₂ values, indicating increased entanglement of the hairpin structure by collagen. In addition, the side-chain NH signals of all Trp residues display similar enhancement of ¹⁵N-NMR relaxation, which becomes so large in the presence of unaligned collagen, that their (H,¹⁵N)-HSQC spectra are no longer observable (FIG. 1A, right panel).

NMR experiments and data shown in FIG. 2 further demonstrate the nature of collagen binding by representative polypeptides containing Trp-rich and well-structured linear hairpin motifs. FIG. 2A shows that collagen gelation or formation of polymerized fibrils from acid-solubilized collagen had no dramatic impact on resonance perturbations observed for MH2-wZIP4 alter mixing with unpolymerized collagen. On the other hand, both MH2-wZIP5 and MH2-wZIP6 exhibited a reduced degree of proton NMR signal perturbations after formation of collagen hydrogels from the mixture of the peptides with acid-solubilized collagen (FIGS. 2B and 2C). Very importantly, MH2-wZIP5 showed a more pronounced loss of resonance perturbations as compared to MH2-wZIP6. FIG. 1D and FIG. 2D further demonstrates that it is only the unpolymerized form of collagen that binds to MH2-wZIP4 since NMR spectral perturbations observed for MH2-wZIP4 after mixing with acid-solublized collagen persisted to the full extent after polymerized collagen fibrils were removed from the collagen hydrogel through centrifugation. Most importantly, such proton resonance perturbations of MH2-wZIP4 for the MH2-wZIP4/collagen solution were found to be highly sensitive to the treatment of a collagenase (FIG. 1D and FIG. 2D). In other words, time-dependent degradation of intact collagen by the collagenase gradually eliminates the proton resonance perturbations of MH2-wZIP4, indicating again that intact (or native) collagen is responsible for the induction of NMR perturbations of peptides containing Trp-rich linear hairpin peptides. On one hand, MH2-wZIP4 showed the most pronounced interference with hydrogel formation by collagen as compared with MH2-wZIP5 and MH2-wZIP6. On the other, collagen-induced perturbations on the NMR spectra of MH2-wZIP5 and MH2-wZIP6 are more significantly affected by collagen polymerization (hydrogel formation) and reduction of binding-competent soluble collagen. These data taken together identify MH2-wZIP4 as a polypeptide having the highest capacity of binding unpolymerized soluble collagen.

### Example 3

### Defining the Functional Modularity of Collagen-Binding Moieties

The methods of this invention are illustrated by a sequence dissection of MH2-wZIP4 in relation to the collagen-binding properties of the MH2-wZIP series of peptides containing the Trp-rich hairpin sequences, especially that of trpzip4. Therefore, an N-terminal fragment IRFTDGEWTWDDA of MH2-wZIP4, or MH2-wZIP4-F1 (Table 4) does not respond to collagen binding, as its proton NMR spectrum remains essentially the same free (dotted spectrum of FIG. 3A) or in the presence of collagen (solid lines in FIG. 3A). A further-truncated fragment, GEWTWDDA, or MH2-wZIP4-F2 (Table 4) behaves the same way as MH2-wZIP4-F1 (FIG. 3B), again indicating lack of binding with collagen. On the other hand, the MH2-wZIP4-F3 fragment or GEWTWDDATKTWTWTEGDFEEIPEEYL (Table 5) responds fully to collagen (FIG. 3C) in the absence of the IRFTD moiety (Table 3). Furthermore, only characteristic resonances of the trpzip4 hairpin moiety, i.e. those of residues GEWTWDDATKTWTWTE (Table 2), exhibit differential resonance perturbations in the presence of collagen, which localizes the sequence motif responsible for collagen binding to within the trpzip4 moiety.

Table 4 lists some Trp-rich peptides that do not bind collagen are therefore not useful in the present invention.

TABLE 4

| Name | Source | Sequence |
|---|---|---|
| MH2-wZIP4-F1 | presently | IRFTDGEWTWDDA (PS + NMR) |
| (P4233)(**) | described | (SEQ ID NO: 68) |
| MH2-wZIP4-F2 | presently | GEWTWDDA (PS + NMR) |
| (P4236)(**) | described | (SEQ ID NO: 69) |
| gb1(**) | Cochran | GEWTYDDATKTFTVTE (PS + NMR) |
| | 2001b | (SEQ ID NO: 70) |
| HP7(**) | Andersen | KTWNPATGKWTE (PS + NMR) |
| | 2006 | (SEQ ID NO: 71) |
| Y2Y11- | Takekiyo | SYTWEGNKWTYK (PS + NMR) |
| trpzip1(**) | 2009 | (SEQ ID NO: 72) |
| W4W9- | Wu 2009 | SVTWENGKWTVK (PS + NMR) |
| trpzip2(**) | | (SEQ ID NO: 73) |
| W2W11- | Wu 2009 | SWTVENGKVTWK (PS + NMR) |
| trpzip2(**) | | (SEQ ID NO: 74) |
| YWWY- | Wu 2009 | SYTWENGKWTYK (PS + NMR) |
| trpzip2(**) | | (SEQ ID NO: 75) |

(**)indicates that these have been produced via the respective routes of synthesis (PS) and that their collagen-binding properties have been studied by use of NMR spectroscopy (NMR).

Table 5 lists some representative molecules of the present invention containing collagen-binding hairpin motifs.

TABLE 5

| Name | Source | Sequence |
|---|---|---|
| v4-gp120-ext2(Q77430- | presently | acetyl-STWTWNGSAWTWNEGGK (PS + NMR) |
| 9HIV1) | described | (SEQ ID NO: 76) |
| v4-gp160-ext2(Q6UYR0- | presently | acetyl-STWTWNGTNWTRNDGGK (PS + NMR) |
| 9HIV1) | described | (SEQ ID NO: 77) |
| MH2-wZIP1(**) | WO/2012/ | IRFTDGSWTWEGNKWTWKGDFEEIPEEYLQ |
| | 142696 | (PS + NMR) (SEQ ID NO: 78) |
| MH2-wZIP1GG(**) | WO/2012/ | IRFTDGGSWTWEGNKWTWKGGDFEEIPEEYLQ |
| | 142696 | (PS + NMR) (SEQ ID NO: 79) |
| MH2-wZIP4(**) | WO/2012/ | IRFDTGEWTWDDATKTWTWTEGDFEEIPEEYLQ |
| | 142696 | (PS + NMR) (SEQ ID NO: 80) |
| MH2-wZIP4-F3(**) | presently | GEWTWDDATKTWTWTEGDFEEIPEEYL |
| | described | (PS + NMR) (SEQ ID NO: 81) |
| MH2-wZIP4-F4(P4225) | presently | IRFTDGEWTWDDATKTWTWTEGSFNPRGS |
| (**) | described | (PS + NMR) (SEQ ID NO: 82) |
| MH2-wZIP4-F5 | presently | IRFTDGEWTWDDATKTWTWTEGGC |
| (P4247)(**) | described | (PS) (SEQ ID NO: 83) |
| BRI-TP304 or | presently | IRFTDGEWTWDDATKTWTWTEG |
| MH2-wZIP4-F6 | described | (SEQ ID NO: 84) |
| FL4247(**)(****) | presently | IRFTDGEWTWDDATKTWTWTEGGC-Fluor750 |
| | described | (PS + in vivo) (SEQ ID NO: 85) |
| BRI-TP404 or | presently | IRFTDGEWTWDDATKTWTWTE-GGS-VHpro10 |
| WZ4-GGS-VHpro10(**) | described | (PS + NMR) (SEQ ID NO: 86) |
| BRI-TP404a or | presently | IRFTDGEWTWDDATKTWTWTE-GDFEE-VHprol0 |
| WZ4-GDFEE-VHpro10 | described | (PS + NMR) (SEQ ID NO: 87) |
| WZ4-GGGGSS-VHpro10 | presently | IRFTDGEWTWDDATKTWTWTE-GGGGSS-VHpro10 |
| | described | (PS) (SEQ ID NO: 88) |
| BRI-TP404b or | presently | IRFTDGEWTWDDATKTWTWTE-GGGSS-VHprolO |
| WZ4-GGGSS-VHpro10 | described | (PS) (SEQ ID NO: 111) |
| BRI-TP406 or WZ4- | presently | IRFTDGEWTWDDATKTWTWTE-GGS-VHpro5 |
| GGS-VHpro5 | described | (SEQ ID NO: 89) |
| BRI-T207 (P4223) or N- | WO/2012/ | FQPRPRFTDGEWTWDDATKTWTWTEGDFEEIPEEYLQ |
| terminally extended | 142696 | (SEQ ID NO: 90) |
| derivative of MH2-wZIP4 | | |
| BRI-T404 | WO/2012/ | IRFTD-GEWTWDDATKTWTWTE-GGS- |
| | 142696 | EVQLQASGGGLVQSGDSLRLSCAASGRTFSTYAMGWF |
| | | RQAPGKLREFVGVISSSGYTHYTNSVRGRFTISRDNA |
| | | KNMVYLQMNSLKPEDTAVYYCAAADRRFIATDGKQYD |
| | | YWGQGTQVTVSSLEHHHHHH (SEQ ID NO: 91) |
| CBLH5-GGC(P4447) | presently | STWTWNGTNWTRNDGGC (PS) |
| (**) | described | (SEQ ID NO: 105) |
| FL4447(****) | presently | STWTWNGTNWTRNDGGC-Fluor750 (PS + in |
| | described | vivo) |
| | | (SEQ ID NO: 106) |

(**)indicates that these molecules have been produced via the respective routes of synthesis (PS) and that their collagen-binding properties have been demonstrated by use of NMR spectroscopy (NMR).
(****)these molecules, i.e. FL4247 and FL4447, were used for imaging studies to demonstrate in vivo efficacy of tissue-specific targeting and localization.

The utility of molecules of the present invention is further illustrated by the following polypeptide from Table 5: IRFTD₅GEWTW₁₀DDATK₁₅TWTWT₂₀EGSFN₂₅PRGS (MH2-wZIP4-F4—SEQ NO: 82) with the DFEEIPEEYL (SEQ ID NO: 92) moiety replaced by recognition site. Ser-Phe-Asn-Pro-Arq (or SFNPR (SEQ ID NO: 93)) for thrombin-specific cleavage of peptide substrates, as established in previous studies (Ni 1995). The use of a thrombin-specific sequence such as FNPR (SEQ ID NO: 94) will enable the release of the GS moiety mimicking a drug conjugated to the rest of the peptide at tissue sites with elevated thrombin activity, e.g. within inflamed joints (Gabriela 2009) and under other inflammatory conditions (Morris 1994; Bogatkevich 2011).

The collagen-binding characteristics of MH2-wZIP4-F4 were established using proton NMR spectroscopy (FIG. 3D) as compared to the “mother” molecule, MH2-wZIP4 (FIG. 1A, FIG. 2). In particular, sharp proton resonances remain for certain residues of MH2-wZIP4-F4, especially those of Asn25, Arg27 and Ser29 (FIG. 3D), while most other residues exhibit broadened proton NMR peaks in the presence of collagen as compared to the free peptide. In other words, the sequence segment of residue Ile1 to Gly22 in MH2-wZIP4-F4 has a similar collagen-binding property as observed for MH2-wZIP4 (FIG. 2) and that the thrombin-recognition site FN₂₅PRGS (SEQ ID NO: 95) are again freely exposed similarly to the DFEEIPEEYL (SEQ ID NO: 92) moiety in MH2-wZIP4 or in MH2-wZIP4-F3.

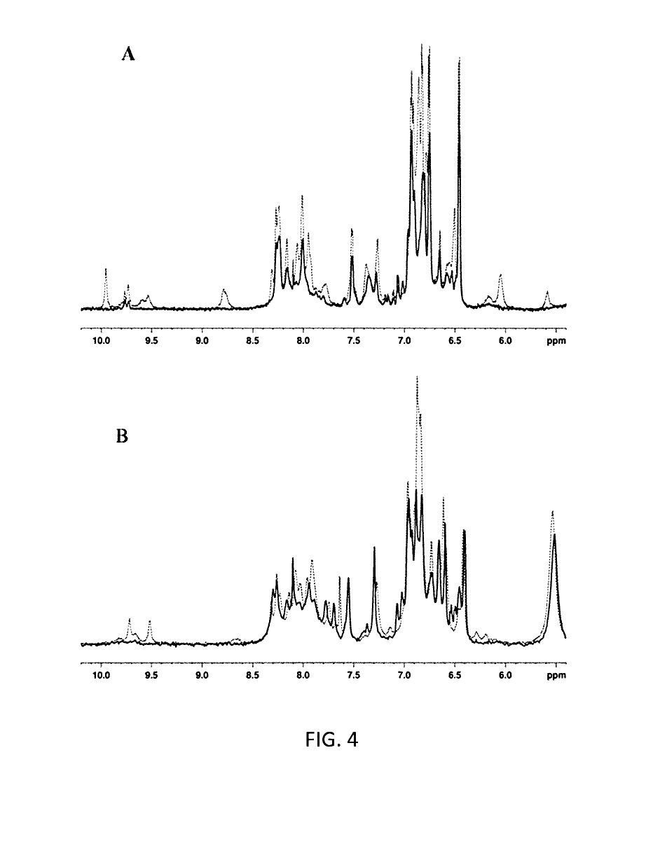

Collagen-binding (or responsiveness) of the trpzip4 motif is also essentially independent of the flanking sequences, as an N-terminally extended analog of MH2-wZIP4 or FQPRPRFTDGEWTWDDATKTWTWTEGDFEEIPEEYLQ (SEQ ID NO: 90—from WO/2012/142696) exhibits a similar pattern of NMR signal perturbations (FIG. 4A). FIG. 4B shows the NMR spectral properties of a peptide MH2-wZIP1GG (i.e. IRFTDGGSWTWEGNKWTWKGGDFEEIPEEYLQ—SEQ ID NO: 79) constructed from the trpzip1 motif (Table 5). It is clear that the trpzip1 moiety in this polypeptide responds similarly to collagen binding as illustrated so far for molecules containing trpzip4, trpzip5 and trpzip6 sequences (FIGS. 1 and 2).

### Example 4

### Hairpin Folding and Binding of Individual Hairpin Peptides to Collagen Determined by Use of High-Resolution NMR Spectroscopy

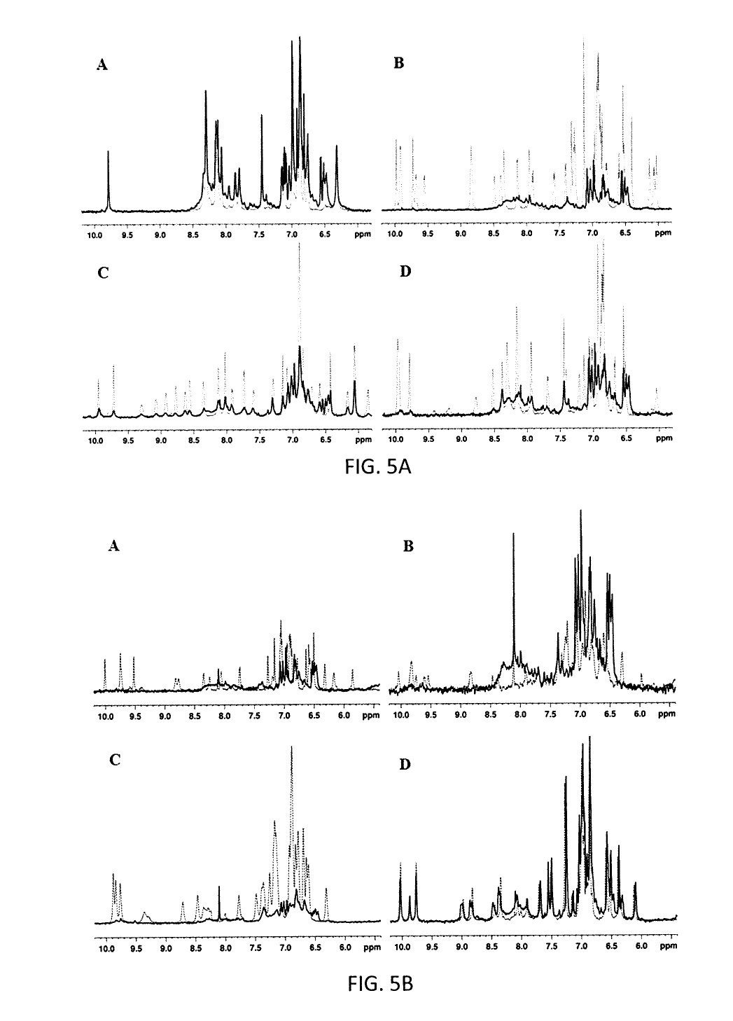

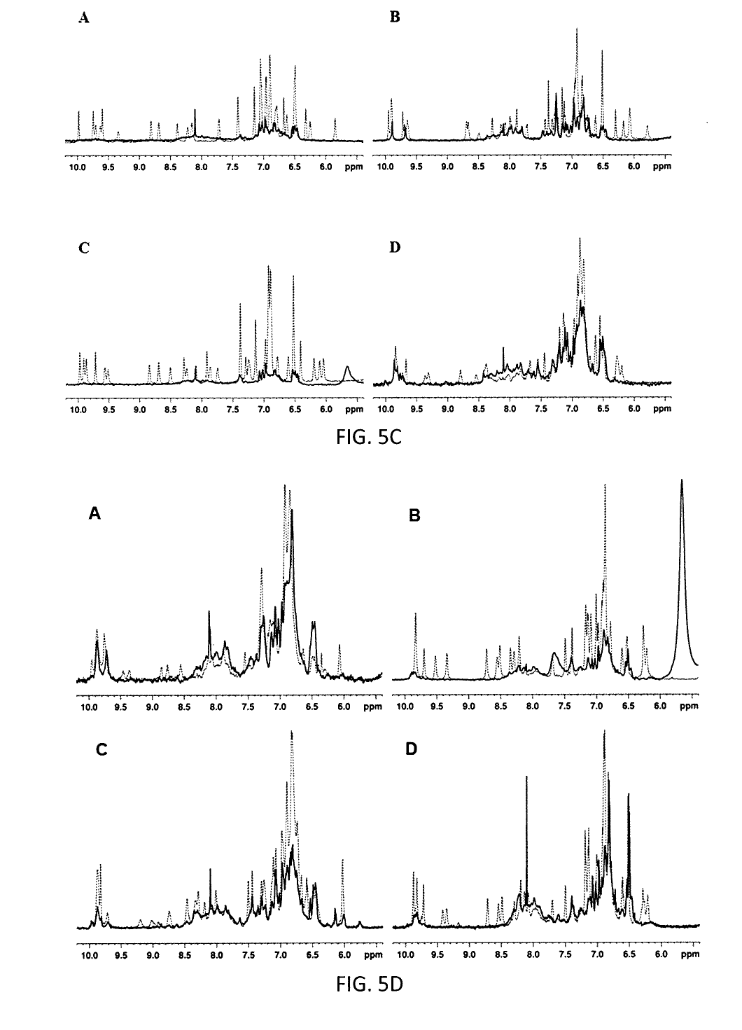

Proton NMR spectroscopy is used to illustrate the folding behavior of the peptides GEWTYDDATKTFTVTE (gb1—SEQ ID NO: 70), GEWTWDDATKTWTVTE (trpzip6—SEQ ID NO: 15), GEWTYDDATKTFTWTE (trpzip5—SEQ ID NO: 16) and GEWTWDDATKTWTWTE (trpzip4—SEQ ID NO: 17) in aqueous solutions. The peptides were synthesized mostly in aminated (R—NH₂) forms and some as C-terminal carboxylate (R—COOH) using standard Fmoc chemistry and purified by use of reverse-phase HPLC. Their identity was confirmed by mass-spectroscopy and NMR spectroscopy. The proton NMR spectra of trpzip4-NH₂, trpzip5-NH₂, trpzip6-NH₂, and gb1-NH₂ (FIG. 5A) illustrate a progressive unfolding or opening of the β-hairpin structure in response to specific amino acid substitutions in these peptides (Cochran 2001b). Therefore, trpzip4-NH₂ exhibits a characteristic hairpin structure, as indicated by the two significantly downfield shifted NH proton resonances between 9.5 to 9.7 ppm (FIG. 5A, Panel B), which belong to residues Thr9 and Thr18, respectively (Cochran 2001b) and by the two overlapped NH signals at 8.85 ppm, which come from residues Trp10 and Trp19. Peptide trpzip5-NH₂ has a less stable hairpin structure, as its NH resonance envelop contracts to start at about 9.3 ppm (FIG. 5A, Panel C) from the 9.7 ppm for trpzip4-NH₂. In addition, the NH resonance of trpzip5-NH₂, especially those between 8.8 and 9.3 ppm have broad line shapes (FIG. 5A, Panel C) which are characteristic of conformational exchanges, here between the closed β-hairpin structure and an opened polypeptide chain. The β-hairpin structure in trpzip6-NH₂ is also less stable since some of its NH resonances, i.e. those between 8.8 and 9.5 ppm (FIG. 5A, Panel D) exhibit very broad lineshapes. These NMR characteristics are in exact parallel with the thermostability of the four hairpin peptides, i.e. with Tₘ about 70° C. for trpzip4, Tₘ about 43° C. for trpzip5, Tₘ about 45° C. for trpzip6 and Tₘ about 7° C. for gb1, as reported previously (Blanco 1994; Cochran 2001b). The conformational characteristics as reflected by the proton NMR spectra are intrinsic properties of the respective hairpin peptides, since the folding behaviors are insensitive to the nature of the C-terminus, i.e. as C-terminal carboxylates or amines (NMR spectra not shown) and all four peptides, i.e. gb1-NH₂, trpzip4-NH₂, trpzip5-NH₂ and trpzip6-NH₂ show the same NMR spectral signatures (FIG. 5A) whether the sample solutions contain a modified PBS buffer (supplemented by 50 mM Na₂HPO₄) or are prepared in 50 mM Tris-HCl, 100 mM NaCl, 0.1% PEG-8000 at pH 7.6 (spectra not shown).

Peptide GEWTYDDATKTFTVTE (gb1—SEQ ID NO: 70) also has little binding capacity nor change of its conformation in the collagen hydrogel (FIG. 5A, Panel A), neither its presence impacts the gelation process of collagen since the degree of gel alignment as measured by the deuterium coupling constant remained similar with or without the peptide (Ni 2012). In sharp contrast, peptide trpzip4 (GEWTWDDATKTWTWTE—SEQ ID NO: 17) is dramatically altered by collagen (FIG. 5A, Panel B), which in the presence of trpzip4, exhibits a greatly-reduced capacity to gelate and a significantly reduced degree of collagen alignment. Both trpzip5 and trpzip6 also respond to the presence of collagen (FIG. 5A, Panel C and D), and the spectral changes of trpzip5 and trpzip6 induced by collagen revert toward those of the free peptides after collagen polymerization (gelation), similar to what were observed for MH2-wZIP5 and MH2-wZIP6 containing the trpzip5 and trpzip6 sequences (FIG. 2). Furthermore, the trpzip5 peptide in the collagen solution reverts to a greater extent as compared to trpzip6 toward the free state after collagen polymerization (NMR spectra not shown), which is paralleled by similar observations with the larger peptides MH2-wZIP5 and MH2-wZIP6 (FIG. 2 and Example 2). These NMR results demonstrate specific, but differential, interactions of Trp-rich hairpin peptides with collagen as illustrated by peptides trpzip4, trpzip5 and trpzip6 containing 4, 2 and 3 Trp residues, respectively (Table 2).

FIG. 5B shows the proton NMR spectra of a shorter trpzip peptide trpzip1 and two predicted hairpin peptides based on the v4-loops of the HIV envelope proteins gp120 and gp160 (Table 2). The trpzip1 peptide and v4-gp120 have characteristic proton NMR spectra of a well-folded hairpin structure (see FIG. 5A) as reported previously (Cochran 2001b). Both peptides also respond to collagen (FIG. 5B, panel A and panel B) similarly as the well-structured trpzip4 (FIG. 5A, panel B) since both contain the WTW sequence motif on both strands of the β-hairpin. Even with the second WTW motif replaced by WTR, v4-gp160 (Table 2) also exhibits proton NMR spectral characteristics of a hairpin peptide responsive to the presence of collagen (FIG. 5B, panel C), which indicates that the WTR motif preserves the function of WTW in hairpin structure formation and in collagen binding. In sharp contrast, the well folded short hairpin peptide HP7 (Anderson 2006) does not respond to the addition of collagen (FIG. 5B, panel D), which indicates that the sequence of HP7, KTWNPATGKWTE (SEQ ID NO: 71—Table 4). is not suitable for collagen binding. As expected, trpzip2 with the sequence of SWTWENGKWTWK (SEQ ID NO: 19) is again a collagen-binding linear hairpin (CBLH) peptide since its proton NMR spectra (FIG. 5C, panel A) are similar to all other CBLH peptides trpzip1, trpzip8 and HP5W4 (FIG. 5C, Panel B, C, D). Collagen-binding characteristics of HP5W4 (FIG. 5A, panel D) is of particular relevance since it shows that the hairpin-stabilizing loop sequence NPATGK (SEQ ID NO: 14) and potentially other loop sequences (Anderson 2004) are compatible with collagen binding as long as the two flanking strands contain Trp-rich sequences W₁X₁X₂ (SEQ ID NO: 1) and X₁X₂X₃ (SEQ ID NO: 2), respectively, especially the WT(W/Y) and WT(W/R) motifs.

FIG. 5D shows the proton NMR spectra of four new peptides based on the amino acid sequence of the gb1 peptide (Table 4, SEQ ID NO: 70) which does not bind collagen (FIG. 5A). Therefore, the CBLH1 variant of gb1 has the amino acid sequence GEWTYNPATGKWTWTE-(NH₂) (Table 2, SEQ ID NO: 94) which contains the WTY and WTW motifs as found in Y4-trpzip1 (Table 2, SEQ ID NO: 22). CBLH2 with a sequence of GEWTWNPATGKWTRTE-(NH₂) (Table 2, SEQ ID NO: 95) incorporates the WTW and WTR motifs found with the collagen-binding peptides V4-gp160 and V4-gp160-ext (Table 2, SEQ ID NO: 26 and 27). Both CBLH1 and CBLH2 incorporate the better-structured turn motif NPATGK (SEQ ID NO: 14) (Fesinmeyer 2004) which result in a better-defined hairpin structure for these peptides according to their proton NMR spectra. Replacement of the first residue Gly in these peptides by a Lys gives us two new peptides KEWTYNPATGKWTWTE-(NH₂) (CBLH3) and KEWTWNPATGKWTRTE-(NH₂) (CBLH4) (Table 2, SEQ ID NO: 98 and SEQ ID NO: 99, respectively). All four new peptides exhibit almost identical collagen-binding characteristics based on proton NMR spectral perturbations as found with trpzip4 and its derivatives (FIG. 5A). The two peptides CBLH3 and CBLH4 contain in addition a substitution of a Lys residue to replace Gly at the N-terminus, conferring additional conformational stability to the hairpin structure (Huyghues-Despointes 2006). However, all these new derivatives of gb1 exhibit a certain degree of reversibility for binding to monomeric collagen, since gelation of the corresponding collagen-peptide complexes was always associated with restoration of the peptide proton NMR spectra as seen with peptides containing trpzip5 and trpzip6 sequences (FIG. 2). Regardless, it can still be concluded that WTY can replace the WTW motif in the first flanking sequence and the WTR motif can replace the WTW motif in the second flanking sequence defining collagen-binding linear hairpin (CBLH) peptides. Furthermore, residues NPATGK can replace DDATKT or other turn-forming residues along with a Lys residue at the N-terminus to stabilize the three-dimensional structure of CBLH peptides without affecting significantly the collagen-binding characteristics.

In the short (12 aa) trpzip peptide (such as trpzip2, Table 2), a well-defined hairpin structure is maintained even after two of the (inner) Trp residues are replaced by Tyr, as in the WYYW-trpzip2 peptide, SWTYENGKYTWK (SEQ ID NO: 23—Wu 2010). Titration with collagen (FIG. 5E, panel C) shows that this WYYW-trpzip2 hairpin retains a significant capacity for binding collagen, which indicates the adequacy of the two motifs, WTY and YTW for hairpin stability and for collagen binding. Such W-to-Y-substitutions also work for the trpzip1 hairpin as both Y4-trpzip1 and Y4Y9-trpzip1 (Takekiyo 2009) retain the collagne-binding capacity (FIG. 5E, panel A, B). As another contrast, the W2W11-trpzip2 variant (Wu 2009) loses the collagen-binding capacity (FIG. 5E, panel D), showing that a valine residue can not replace the functional W or Y residues (i.e. not from W₁T₁(W₁/Y₁)ₜₒ W₁T₁V₁ and from W₂T₂W₂ to V₂T₂W₂) in maintaining the collagen-binding property of a hairpin peptide.

The putative hairpin sequence, TWRWNGSTAWTWS (i.e. SEQ ID NO: 28—pp-D7EUT8, Table 2) based on a predicted protein in Mycobacterium tuberculosis does not appear to fold into a stable hairpin structure since its proton NMR spectrum is somewhat broad (spectrum not shown). However, some proton NMR perturbations are observed for pp-D7EUT8 after titration with collagen (data not shown). Such less stable hairpin folding is likely due in part to the non-optimal 5-residue turn sequence NGSTA, as compared to the four-residue ones in the V4 hairpin loops of HIV proteins gp120 and gp160 (Table 2). Further stabilization of the hairpin structure may require the addition of N- and C-terminal extensions based on the predicted protein sequence, i.e. from TWRWNGSTAWTWS (SEQ ID NO: 28) to STWRWNGSTAWTWSTAS (SEQ ID NO: 29—Table 2). Such sequence extensions will also be required for hairpin stabilization of the peptides derived from the V4 loops of other variants of the HIV1 envelope protein gp120, i.e. v4-gp120v2, pp-Q77413, pp-Q77425 and pp-Q904S5 (Table 2). In this regard, the two extended and derivatized peptides, acetyl-STWTWNGSAWTWNEGGK (SEQ ID NO: 76—Table 5) and acetyl-STWTWNGTNWTRNDGGK (SEQ ID NO: 77—Table 5) were shown to bind to type-I collagen by use of proton NMR spectroscopy (data not shown), similarly to other CBLH peptides (FIGS. 3. 4. and 5). More importantly, binding of acetyl-STWTWNGSAWTWNEGGK (SEQ ID NO: 76—Table 5) to collagen is essentially irreversible since it competes effectively with collagen polymerization (data not shown) as observed with CBLH peptides containing trpzip4 (FIG. 2). On the other hand, a certain degree of reversibility is observed for collagen binding of acetyl-STWTWNGTNWTRNDGGK, a property quite similar to the CBLH peptides trpzip5, trpzip6 and other derivatives of the non-functional gb1 sequence CBLH1, CBLH2, CBLH3 and CBLH4 (Table 2).

### Example 5

### Collagen-Binding Properties of Trpzip4-Protein Conjugates

The collagen-binding capacity of Trp-rich hairpin peptides was further illustrated through a recombinant protein containing MH2-wZIP4-F6/BRI-TP304 with a sequence of IRFTD₅GEWTW₁₀DDATK₁₅TWTWT₂₀EG (SEQ ID NO: 84—Table 5) at the N-terminal region:

(SEQ ID NO: 86 - BRI-TP404)
IRFTD₅GEWTW₁₀DDATK₁₅TWTWT₂₀EG-GS-[VHpro10]

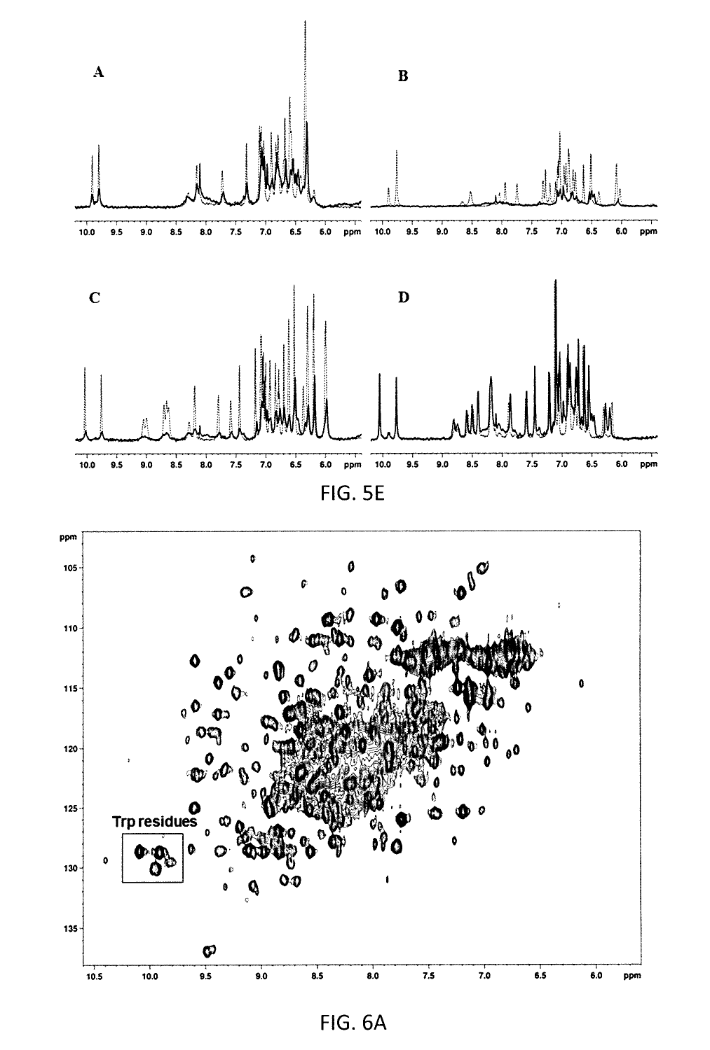

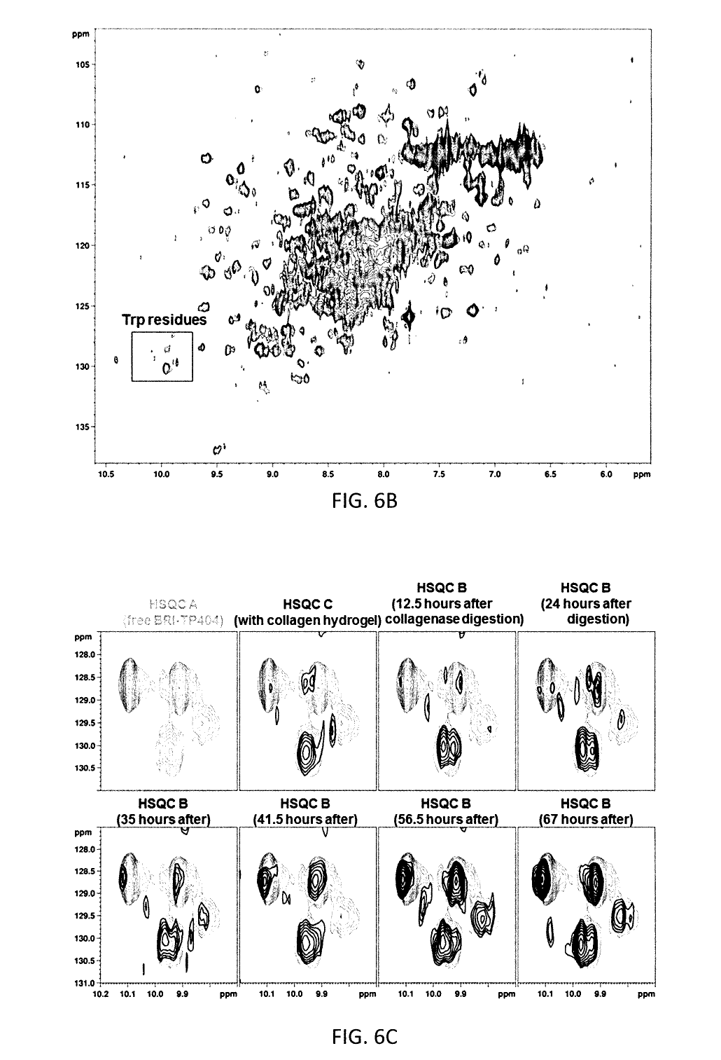

Here, VHpro10 is an antibody VHH fragment in the form of a prothrombin-specific ligand. The collagen-binding properties of BRI-TP404 or WZ4-GGS-VHpro10 (Table 5) are characterized by use of NMR spectroscopy similarly to what was carried out with MH2-wZIP4 (FIG. 1). Recombinant BRI-TP404 with uniform labeling of the ¹⁵N isotope allowed the identification of the four Trp residues unique to the trpzip4 region (FIG. 6A). These four Trp residues responded similarly to collagen binding (FIG. 6B) as the same residues in MH2-wZIP4 (FIG. 1A). In contrast, practically all residues of the VHpro10 portion of BRI-TP404 are fully exposed when the targeting portion (i.e. the N-terminal sequence carrying the trpzip4 domain) gets adsorbed on the collagen matrix. In other words, BRI-TP404 exhibits a similar collagen binding characteristics as MH2-wZIP4, demonstrating that the targeting capacity of the trpzip4 motif and other collagen-binding linear hairpins is relatively independent of the nature of the C-terminal extension or the payload protein VHpro10.

The binding specificity of BRI-TP404 was further determined through the dissolution of the collagen (gel) matrix using enzymes that cleave and convert the triple-helical collagen into small peptides. FIG. 6C shows that the sequence segment of BRI-TP404, especially the four telltale Trp residues, gradually came into view while the collagen gel dissembles in the presence of a collagenase. In the same timeframe of several days, the VHpro10 portion of the BRI-TP404 remains unchanged as compared to the starting point where the entire protein is associated with the collagen matrix through the targeting moiety in BRI-TP404. As a comparison, collagen-adsorbed MH2-wZIP4 (FIG. 1D) is also releasable by the collagenase in exactly the same timeframe as for the dissolution of the collagen matrix (FIG. 1D). In all these experiments, destruction of the collagen hydrogel is evidenced by the conversion of a translucent liquid gel into a clear fluid characteristic of aqueous solutions of peptides and proteins.

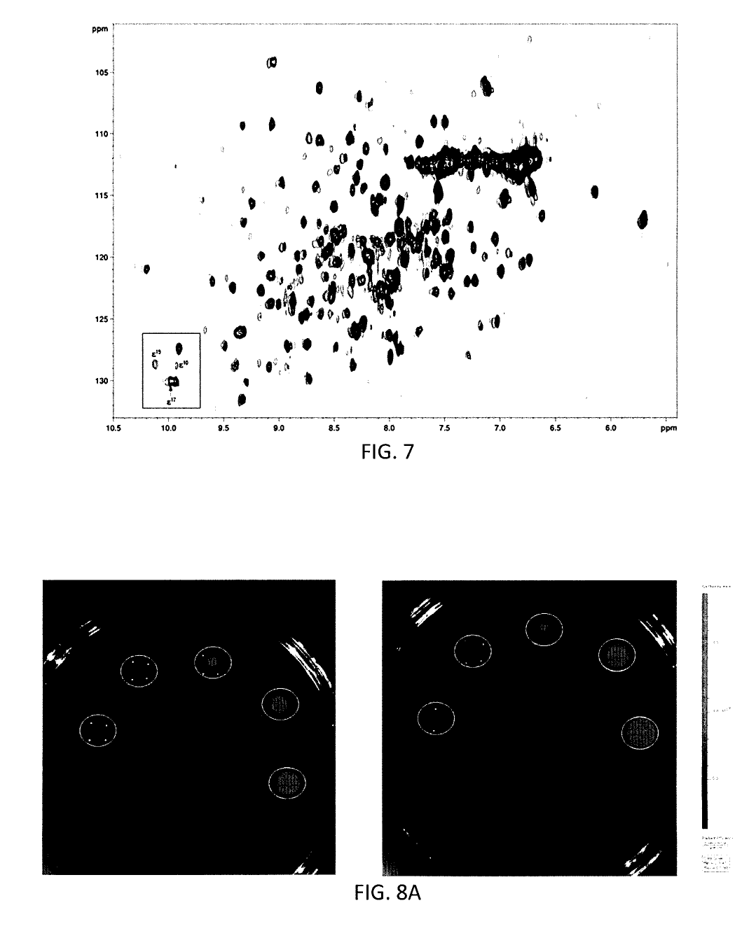

Similarly to the CBLH-conjugated protein BRI-TP404, our previous patent publication (Ni 2012) demonstrated the collagen-binding capacity of another antibody VH10 protein conjugated to the peptide IRFTDGEWTWDDATKTWTWTEG (SEQ ID NO: 84) at its N-terminus to form BRI-T404 (SEQ ID NO: 91) (Table 5). The relevant NMR data are reproduced here as FIG. 7 to illustrate the universal (or context-independent) utility for CBLH peptides.

### Example 6

### Conjugates of Fluorescence Probes, Small-Molecule Drugs and Polymers with Collagen-Binding Hairpin Peptides

In the absence of cysteine (Cys) residues (or disulfide bonds), collagen-binding hairpin peptides can accommodate the addition of a Cys residue as either an N-terminal or a C-terminal extension. An example is MH2-wZIP4-F5 (Table 5), which contains the collagen binding trpzip4 with an N-terminal extension of IRFTD and with a Cys residue added to the C-terminus through a two-residue (GG) connecting sequence. The corresponding peptide, P4247, was synthesized using standard Fmoc chemistry and purified by use of reverse-phase HPLC. Its identity was confirmed by mass-spectroscopy and NMR spectroscopy. This Cys-containing peptide, i.e. P4247, can be coupled to a fluorescent probe, a small-molecule drug or a polymer support following the well-established thiol-maleimide conjugation chemistry (Ghosh 1990; Brinkley 1992). Therefore, a fluorescent dye such as the HiLyte Fluor-750 C2 maleimide (AnaSpec, California) or DyLight(DL) Fluor-755 maleimide (Thermo Scientific) was first prepared at a concentration of ˜4 μM in 2 mL of degassed ˜0.2 M HEPES, 1 mM EDTA, pH 7.7. To this solution of the fluorescent probe was added ˜0.2 mL of a 0.2 mM solution of the thiolated peptide P4247 (˜40 nmol) in 25 mM sodium borate, pH 9. The reaction mixture was kept under an argon atmosphere and the conjugation was allowed to proceed for 16 h at 23° C. Depending on the nature of the collagen-binding peptide, the fluorescent probe containing the maleimide functional group can also be in molar excess (5-10 fold) as compared to that of the Cys-modified peptide, following essentially the experimental procedure as reported previously (Ghosh 1990; Mushero 2011). The peptide-probe conjugate was separated and purified by use of HPLC and identified by use of mass spectrometry and NMR spectroscopy. The purified material was dried by lyophilization and reconstituted in pure water at a concentration between 0.2 mg/mL and 1 mg/mL before use.

Carboxyl- and amine-terminated solid supports are available commercially and can be activated for coupling to the free —SH group of a Cys-modified CBLH peptide. For example, an NH₂-terminated dextran coating can be modified with the N-succinimidyl 3-(2-pyridyldithio)-propionate reagent (SPDP). The SPDP reagent adds disulfide-containing linkages that can be cleaved with reducing agents for final conjugation to sulfhydryl-containing peptides through a disulfide bond. On the other hand, the carboxy-terminated dextran coating can be modified through maleimide coupling with succinimidyl 4-[N-maleimidomethyl]cyclohexane-1-carboxylate (SMCC) and 1-ethyl-3-[3-dimethylaminopropyl]carbodiimide Hydrochloride (EDC) to create a non-labile thioether bond between the peptide and the solid support.

Polymers conjugated to collagen-binding hairpin peptides can generally be synthesized through a selective amine coupling procedure employing water-soluble carbodiimide chemistry (Lee 2008; Lee 2012). Here, collagen-binding peptides are first extended at the N-terminus, e.g. via a four-residue (SEC) ID NO: 96) or G4 linker sequence or alternatively to include the five-residue sequence IRFTD (SEQ ID NO: 53) as the linker as in the case for BRI-TP304 or MH2-wZIP4-F6 (Table 5). Solutions of such modified collagen-binding peptides are mixed with or loaded onto a polymer containing free carboxylic groups, one example of which is the anionic GRAS (Generally-Regarded As Safe) polysaccharide alginate (Lee 2012) or carboxylated dextran matrices. Other examples for conjugation with collagen-binding peptides are lipid micelles decorated with free carboxylic groups, such as the vitamin E derivative D-α-tocopheryl polyethylene glycol succinate (TPGS) (Zhang 2012) or a structurally-related analog D-α-tocopheryl polyethylene glycol sebacate (Lipshutz 2008). To hydrated polymers or lipid micelles is added a freshly mixed solution containing N-ethyl-N′-(3-diethylaminopropyl) carbodiimide (EDC) and N-hydroxysuccinimide (NHS) with a controlled reaction times for potentially selective coupling (Lee 2008) to the free N-terminal amines of the modified collagen-binding peptide. Alternatively, insoluble polysaccharide polymers can be activated by oxidation with periodate (Sanderson 1971) to produce reactive aldehydes for amine-reactive coupling as utilized for the synthesis of peptide-conjugated polysaccharide vaccines (Lett 1995; Lett 1994) and for the fabrication of biocompatible soft tissue adhesives (Mo 2000; Wang 2007; Mandevi 2008). In this regard, the collagen-binding v4-gp120 and v4-gb160 peptides (Table 2) are of particular utility since the absence of Lys residues enables selective coupling via an added Lys to N-terminally-acetylated peptides. In other words, both v4-gp120 and v4-gp160 can be acetylated at their N-termini and extended at their C-termini to contain an added Lys residue, resulting in the modified CBLH peptides, acetyl-STWTWNGSAWTWNEGGK (SEQ ID NO: 76—Table 5) and acetyl-STWTWNGTNWTRNDGGK (SEQ ID NO: 77—Table 5), which can be used for selective conjugation with GRAS polysaccharides or caboxylated drug delivery vesicles. Such GRAS substances conjugated with collagen-binding hairpin peptides, or CBLH-GRASPs in short, can be used as tissue-specific vehicles for localized drug delivery to fibrotic/fibrosing tissues as previously demonstrated for other collagen-binding peptides (Rothenfluh 2008; Setton 2008; Chan 2010) and for naturally-occurring polysaccharides derivatized with cell-specific binding capacities (Lee 2012). We further envision the fabrication of blends of CBLH-GRASPs with unpolymerized collagen, which may increase the retention and localization of drug delivery vehicles through a “seemless” integration with native collagen accumulated at sites of tissue inflammation and fibrosis.

Collagen-binding peptides can also be conjugated to magnetic nanoparticles through covalent chemistry. Two types of dextran-coated magnetic nanoparticles are used for covalent conjugation, one MNP-CO₂H with free carboxylates and the second MNP-NH₂ with free amines. 10 mg (300 nmols) (at 10 mg/ml) of MNP-CO₂H is activated for 15 minutes by addition of EDC at 0.6 mg (3 μmols)/60 μl H₂O and sulfo-NHS at 1.73 mg (15 μmols)/200 μl H₂O before addition of the CBLH peptide at 1.5 μmol/150 μl of 25% CH₃CN in H₂O. The reaction is allowed for 2 hours before the MNP-peptide product is concentrated. 10 mg (300 nmols) (at 10 mg/ml) of MNP-NH₂ is activated for 30 minutes by addition of SM(PEG)₄ at 4 μl (250 mM)(1 μmol) before addition of the CBLH peptide with a free thiol group at 1 μmol/100 μl in 25% of CH₃CN in H₂O. The reaction is allowed for 30 minutes before the MNP-peptide product is concentrated.

### Example 7

### Hydrogel Formulations of Collagen-Binding Compounds Demonstrated by Use of a Fluorescent Trpzip4 Peptide

Collagen-binding hairpin peptides can be used to create hydrogel formulations of drugs, peptides or therapeutic proteins for localization of their pharmaceutical action, such as and especially for tissue engineering applications (Han 2009; Sun 2009). In this regard, collagen-based hydrogels are re-emerging as biomaterials in regenerative medicine due to the abundance of collagen in the animal kingdom and the low antigenicity, biocompatibility and biodegradability of collagen-based biomimetic scaffolds (Abou Neel 2012). Of particular importance is our experimental finding (FIGS. 1 and 2 and Example 2) that compounds bearing collagen-binding hairpin (CBLH) peptides are highly specific for monomeric and unpolymerized collagen and appear to have little affinity for polymerized collagen. This important property of CBLH peptides makes it possible to prepare liquid formulations of drug-loaded collagen that can be used either alone or for reformulation with other polymer-based drug carriers or delivery vehicles, such as the GRAS (Generally-Regarded As Safe) polysaccharides or lipid micelles (see Example 6).

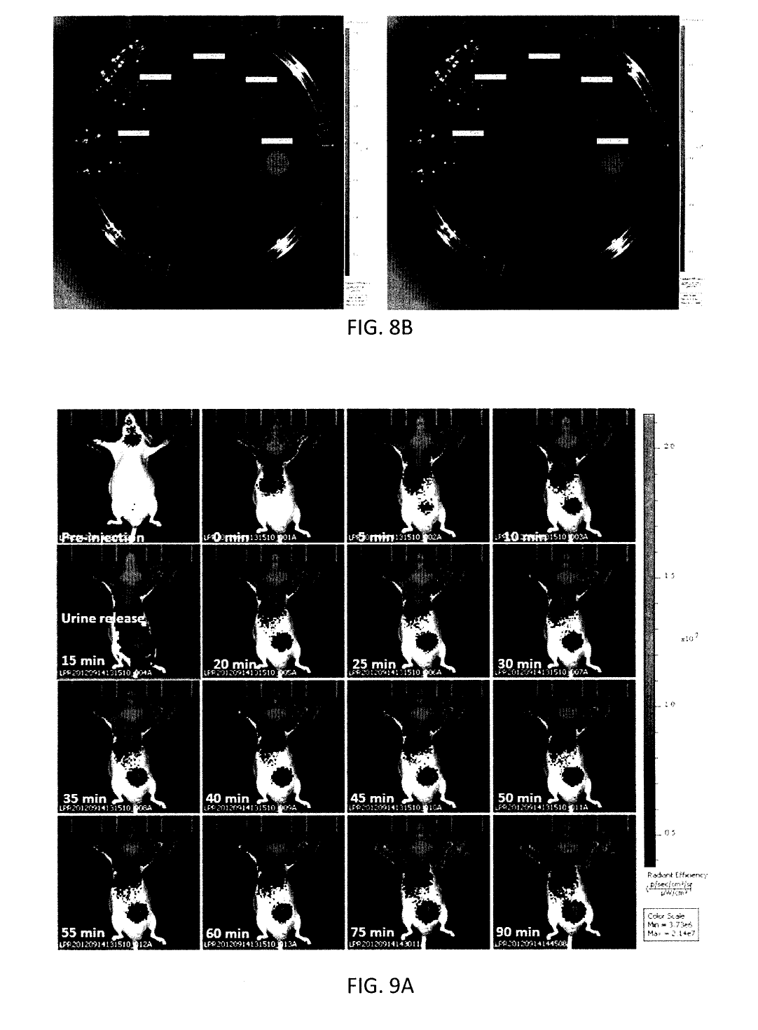

Peptide P4247 conjugated to a fluorescent probe, i.e. FL4247 (Table 5), was therefore used together with rat-tail collagen (type I) to assess the binding capacity of CBLH peptides to collagen and to illustrate the potential applications of collagen hydrogels formulated with compounds of this invention. The fluorescent probe, HiLyte Fluor750 (AnaSpec, California) was selected with a maximum absorption at an optical wavelength of 750 nm and with a maximum fluorescence emission at 800 nm. Therefore, the intensity of fluorescence emissions would also be optimal for in vivo imaging of tissue locations with a differential enrichment of the injected fluorescent compound (see Examples 8-10). Here, small disks of collagen hydrogels were formed in a petri dish following the established protocols for making randomly-deposited collagen hydrogels (see Example 2). For this first experiment, FL4247 in a hydrogel solution were loaded onto the pre-formed collagen hydrogels and molecular diffusion off FL4247 was followed by fluorescence imaging (FIG. 8A, left image). For the second experiment, FL4247 solutions were prepared by pre-mixing with collagen (FIG. 8A, right image) and collagen gelation was allowed to proceed in the presence of FL4247 similarly as for NMR-binding experiments (FIGS. 1-2). Such FL4247-collagen hydrogel preparations (FIG. 8A, right) were then submerged in a large volume of buffer solutions (e.g. the PBS) and the degree of FL4247 retention by the hydrogels were followed by fluorescence imaging (FIG. 8B). Looking at FIG. 8A in more detail, the fluorescently labeled peptide FL4247 in a collagen hydrogel displayed the highest fluorescence intensity when 5-10 μL of the peptide/collagen solution (at a concentration of ˜6 μM) was applied to a disk of collagen hydrogel formed from 100 μL of a dilute collagen solution (see Example 2). However, the observed fluorescence intensity was still restricted close to the center of the hydrogel disks 30 minutes after the FL4247 solution was applied (FIG. 8A, left). FIG. 8B shows the fluorescence images of hydrogel solution premixed with FL4247 at approximate concentrations of 0, 60, 180, 300 and 600 nM (or in the same volume ratios of 0:100, 1:100, 3:100, 5:100 and 10:100) of the FL4247 solution at 6 μM as compared to the collagen (EMPI) solution. Due to premixing, fluorescence intensities were essentially uniform across the entire disks of the collagen hydrogel, which persisted at decreased levels one hour after addition of a buffer solution submerging the hydrogel disks. The imaging data therefore show that pre-formed hydrogels significantly retarded the diffusion of FL4247 in the gel matrix (FIG. 8A, left and right panels) and uniformly distributed FL4247 in the gel matrix (by pre-mixing) could resist outward diffusion into buffer solutions (FIG. 8B). These observations were in exact agreement with the NMR-based binding data that show a dramatic alteration of the proton NMR spectra of the trpzip4 peptide (FIG. 5) and compounds containing the trpzip4 sequence as the covalently-linked collagen-binding moiety (FIGS. 2-4, 6, 7).

### Example 8

### In Vivo Biodistribution of a Collagen-Binding Hairpin Peptide Determined by Fluorescence Imaging

Immune-compromised nude mice (20-30 g) and normal Sprague-Dawley rats (250-310 g; Charles River Laboratories, St-Constant, QC, Canada) were acclimated for at least 3 days prior to the start of the study. Animals were housed in microisolator cages and were kept on a 12-hr light/dark cycle with constant temperature and humidity. Food and water were provided ad libitum. Stock solutions of various agents and peptides of this invention were diluted in saline (0.9% sodium chloride) prior to use. Saline formulations of FL4247 were prepared in such a way that similar dose levels were achieved based on the body weight of the experimental animals used for the study (i.e. in mg/kg).

Of the different methods available, imaging with fluorescently-labeled test compounds is particularly useful for assessing the in vivo biodistribution in real time with live animals. Therefore, the biodistribution of FL4247 was followed over time in two nude mice each for two routes of administration: 1) intravenous (I.V.); and 2) intraperitoneal (I.P.). Another imaging experiment was carried out using a Sprague-Darley rat (Charles River Laboratories, St-Constant, QC, Canada) through a tail-vein (I.V.) administration of an FL4247 solution.

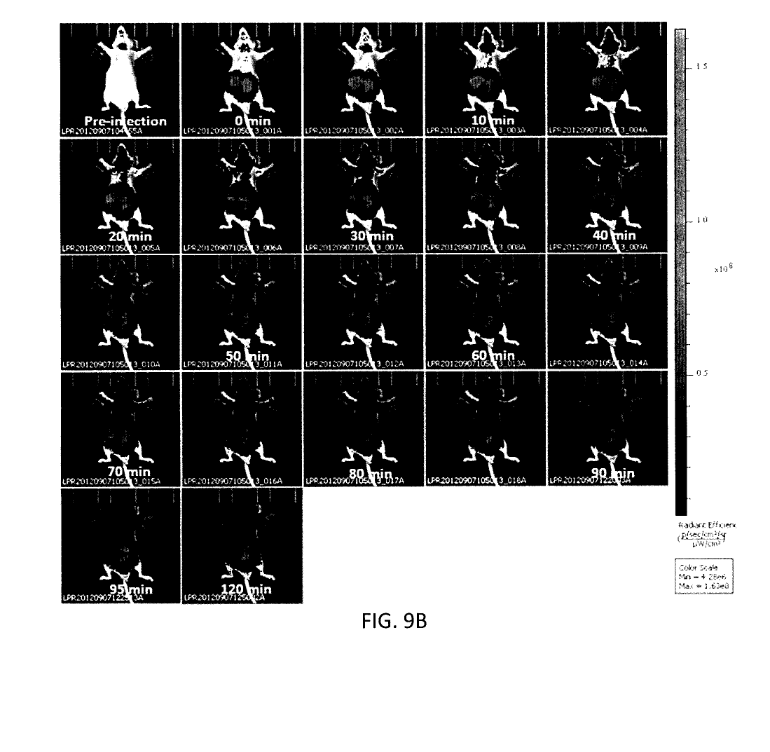

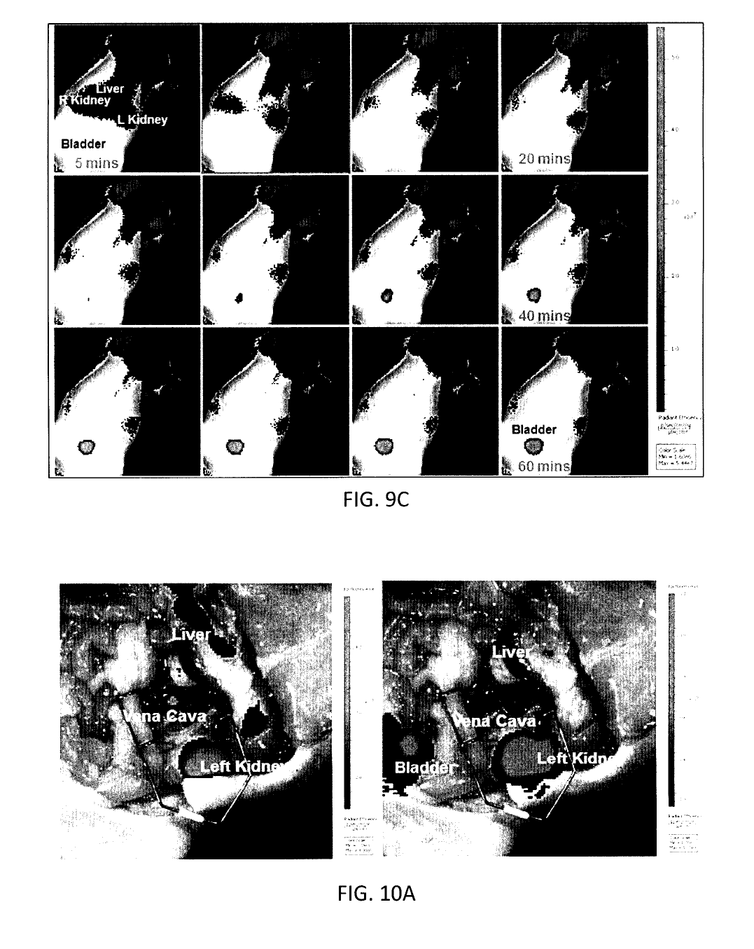

The I.V. injection route depicted similar results in both studies even though the imaging sequence parameters were different (study 1=1 image/2 minutes over 30 minutes; study 2=1 image/5 minutes over 60 minutes). Immediately following injection, FL4247 accumulated rapidly in the head region followed by a slow decrease of fluorescent intensity over time (FIG. 9A). FL4247 also accumulated in the bladder with the fluorescence intensity steadily increasing over time. Beside the head and the bladder, no other organs appeared to accumulate or retain FL4247 in these healthy nude mice used for these studies.

Similar results were obtained in two studies involving the I.P. route of injection. Different imaging parameters were again used (study 1=1 image/2 minutes over 60 minutes; study 2=1 image/5 minutes over 90 minutes). The I.P. injection of FL4247 led to the accumulation of the compound at the level of the head within 5 minutes. However, the total accumulation of the fluorescent compound was far inferior over time compared to the I.V. injection. The distribution of FL4247 over the entire body is complete within 30 minutes accompanied by a slow accumulation of fluorescence in the bladder region. Besides the bladder, no specific local accumulation of FL4247 was noted in both studies.

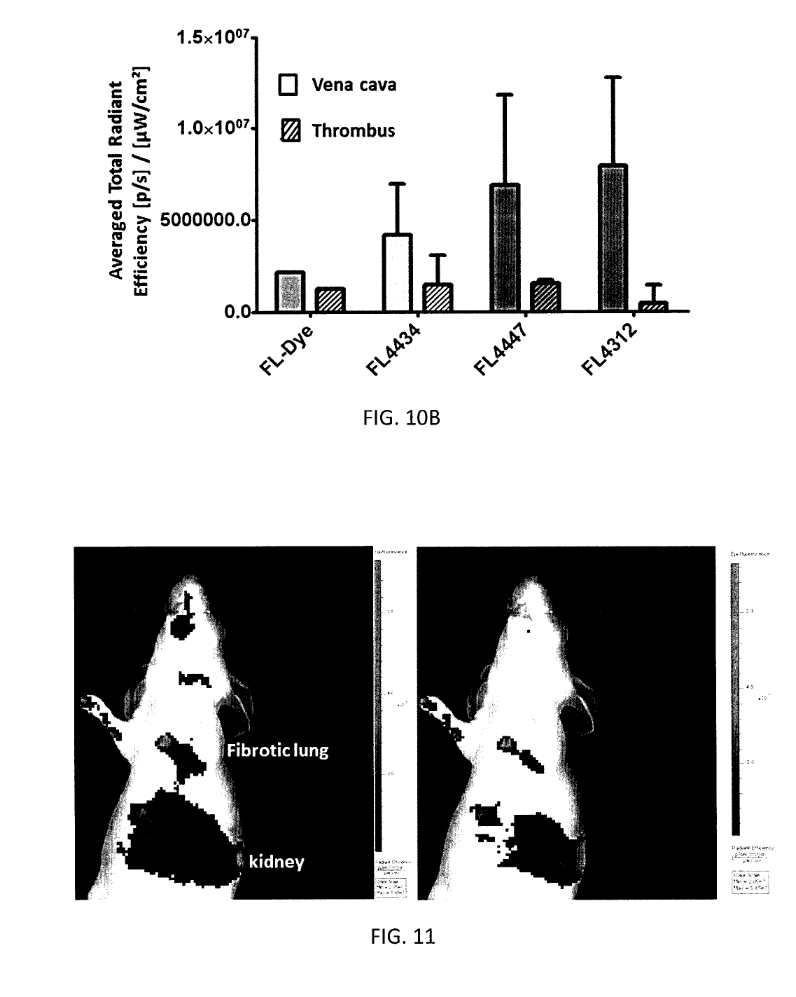

The biodistribution was also assessed using one rat in which FL4247 was injected intravenously (I.V.) through the tail vein. To illustrate the capacity for image contrast, only 10% (one tenth or 1/10) of FL4247 was used with the normal rat (FIG. 9C) as compared to the dosage of FL4247 used for imaging vascular injury in venous thrombosis (see FIG. 10). Even at such a reduced dose, fluorescence imaging with FL4247 can locate its differential accumulation in the head region, in the kidneys and in the bladder (FIG. 9C). The fluorescence data (FIG. 9C) revealed first an accumulation of the compound in the liver (the head was not in the imaging field) that decreased rapidly (10 times within 5 minutes). Then the fluorescence became more intense in both kidneys for the next 25 minutes and finally the fluorescence appeared in the bladder for the remainder of the experiment time. Although the head was not imaged during this experiment, ex vivo imaging was carried out at the end of the live-animal protocol with the dissected brain, heart, liver and both kidneys. A small amount of fluorescence was detected in the liver and a much stronger signal (about 10 times) was present in both kidneys. Nothing was detected in the brain and heart organs, an important result considering the in vivo data of the I.V. injected mice where there was accumulation of the compound at the level of the head. Therefore, FL4247 detected in the head region does not appear to remain in the brain, but instead very likely a result of local retention in the oral/nasal cavity outside of the brain.

### Example 9

### Imaging Inflamed Vasculatures Using Rat Models of Venous Thrombosis

Accumulation of morphologically-diverse collagen structures is a hallmark of atherosclerotic inflammation and unstable atherosclerotic plaques (Sukhova 1999; Penz 2005; Adiguzel 2009; Reininger 2010) as well as fibrotic diseases in general (Wynn 2004). Such de-structurization of aligned tissues is also mimicked by some animal models of vascular injury induced by FeCl₃ application to blood vessels (Eckly 2011). Trp-rich hairpin peptides are shown here to respond to unpolymerized type-I collagen and to interfere with the formation of hydrogels by type-I collagen, which mimic the physicochemical environment of normal and inflamed tissues (Houdijk 1985; Eckly 2011; Torbet 2007). The application of ferric chloride on venous and arterial vessels was therefore the method used to produce the inflamed vasculature in the animal model. The carotid artery (arterial model) and the abdominal vena cava (venous model) were the chosen vessels for the imaging experiments with injured blood vessels undergong thrombosis.

The FeCl₃ rat venous model of thrombosis was generated as described (Wang 2005) with some modifications. Briefly, rats were anesthetized with a 2.5% isoflurane/oxygen mixture and placed on a heat source (35-37° C.). The vena cava was then exposed via a midline incision and the region between the renal and iliolumbar veins was isolated. One minute after drug administration, a piece of filter paper (Gel Blot Paper, GB003, Whatman, Piscataway, N.J., USA; 7 mm diameter) saturated with 10% FeCl₃ (EMD Chemicals Inc., Gibbstown, N.J., USA) was placed on the exposed surface of the vena cava and incubated for 3.5 minutes. During the application of FeCl₃, the abdominal region was covered with aluminum foil. At the end of the incubation period, the filter paper was removed and the exposed viscera covered with a saline-soaked gauze.

In a first experiment, FL4247 was injected immediately after ferric chloride application with the imaging session started right after. Whole-body (rat) imaging data did not reveal any fluorescence at the site of vascular injury. However side-by-side ex vivo imaging of the injured abdominal vena cava and the thrombus produced by the vessel injury showed fluorescence retention by the injured vascular wall. In another experiment, a waiting time of 30 minutes was observed between the end of the ferric chloride application and the I.V. injection of FL4247 through the tail vein. In this experiment, whole-body imaging showed the presence of a strong fluorescence signal (FIG. 10A, left) at the location of the injured abdominal vena cava. Such local fluorescence quickly faded away with the increase of fluorescence intensities in the left kidney and in the bladder region (FIG. 10A, right). Ex-vivo imaging of excised organs showed strong fluorescence intensities along the injured vasculature covered by the blood clot. Very interestingly, a much weaker signal was detected in the dissected thrombus (about 3 times), which indicates that FL4247 was able to penetrate the thrombus to reach the vascular wall. Similar experiments were carried out with a four- to five-fold reduction of the injected FL4247, i.e. at a dose level twice that used for the rat biodistribution study (FIG. 4C). This time, whole-body imaging did not reveal fluorescence localization at the site of vascular injury. However, fluorescence intensity showing the presence of FL4247 was again noted in side-by-side imaging experiments of excised blood vessels in comparison with the blood clot (thrombus) (FIG. 10B, Table 6). Finally, we noted a rapid clearance of FL4247 via the kidneys to the bladder that was similar to what was observed in the biodistribution studies (see Example 8).

TABLE 6

| | FL-Dye | FL4434 | FL4447 | FL4312* |
|---|---|---|---|---|
| # of Rats | 2 | 4 | 3 | 4 |
| Mean Body weight (g) | 293 | 275 | 283 | 278 |
| Volume of imaging | 200 μl | 195 μl | 200 μl | 200 μl |
| probe injected | | | | |
| Mean length of FeCl₃ | N/A | 7.37 | 7.65 | N/A |
| injury (mm) | | | | |
| Mean thrombus weight | 0.02155 | 0.02646 | 0.02184 | 0.02793 |
| (g) | | | | |

*FL4312 is structurally identical to FL4247 of FIG. 10A, but prepared from a different synthesis of the starting peptide P4247. These volumes of the injected imaging probes correspond to approximately ¼^{th} of the highest dose of FL4247 used in imaging experiments (see FIG. 10A).

In order to clarify the specificity of FL4247 for tissue localization, two rats with venous thrombosis were imaged following I.V. injection through the tail vein of the unreacted dye purified from the preparation of the fluorescently-labelled compounds (e.g. FL4247). In these imaging experiments, there were also a 30-minute waiting time after introduction of FeCl₃ to the exposed vena cava to ensure the development of substantial vascular injury and the deposition of a thrombus. In contrast to what was observed with FL4247 (FIG. 10A left), no local accumulation of fluorescence was observed following the injection of the dye alone. Most importantly, post-mortem dissection revealed greatly-reduced fluorescence intensities for the dye at the segment of vena cava injured by FeCl₃ as compared to FL4247 (here as FL4312) and to FL4447 (FIG. 10B, Table 6). Similarly, significantly-reduced fluorescence was observed at the injured vena cava (as compared to the thrombus) for a conjugate (FL4434) of the fluorescent dye with peptide GEWTYDDATKTFTVTEGGC based on the non-functional (for collagen binding) gb1 sequence (SEQ ID NO: 70). Very importantly, a somewhat reduced level of local fluorescence as compared to FL4247/FL4312 was observed for a fluorescence conjugate (FL4447, SEQ ID NO: 106) based on the peptide STWTWNGTNWTRNDGGC (SEQ ID NO: 105). This latter localization behavior is apparently related to the observed reversibility of collagen binding for a related peptide acetyl-STWTWNGTNWTRNDGGK (SEQ ID NO: 77), as established through proton NMR spectroscopy.

In summary, fluorescent intensities were retained by the injured blood vessel for rats with venous thrombosis (induced by application of 10% FeCl₃ on the vena cava blood vessel), even at the one-hour time point when most injected FL4247 has cleared through the kidney to the bladder into the urine. Retention of FL4247 was substantial even with a five-fold reduction of the dose of the injected FL4247. In addition, a more elaborated vessel injury along with a blood clot (e.g. that evolved for 30 minutes after application of FeCl₃) retained a higher level of FL4247 fluorescence as compared to FL4247 injection immediately before FeCl₃ application. The observed fluorescence retention with FL4247 appears to be a result of the specific targeting of the CBLH peptide in FL4247, as the dye alone or a conjugate of the dye with a non-functional peptide GEWTYDDATKTFTVTEGGC based on the gb1 sequence (SEQ ID NO: 70) did not accumulate to significant levels in the injured vein.

### Example 10

### Imaging Tissue Inflammation Using a Mouse Model of Pulmonary Fibrosis

Tissue localization of the representative compound FL4247 containing a collagen-binding hairpin peptide was further examined using a bleomycin-induced mouse model of pulmonary fibrosis. After injection through the tail vein, a high degree of FL4247 fluorescence was retained in the area of the lung for fibrotic mice. This is accompanied by fluorescence localization into generally inflamed tissues in the oral cavities of these mice, which are induced through either mechanical or contact damages. It can therefore be concluded that FL4247 has an affinity for inflamed/fibrosing tissues in vivo, very likely due to specific binding of the collagen-responsive hairpin moiety to newly-secreted collagen enriched in these tissue environments.

Specifically, pulmonary instillation of bleomysin was used to initiate pulmonary fibrosis in a mouse model using immune-compromised nude mice. The objective was to determine if the fluorescent FL4247 compound would localize in fibrotic tissues of the lung induced by bleomycin. Three mice with pulmonary fibrosis received FL4247 intravenously and one intraperitonealy at a dose level twice those used for biodistribution studies with mice (FIGS. 9A and 9B) or twice the equivalent dose used in imaging venous thrombosis with rats (FIG. 10). The I.V. injection route produced more clear co-localizing results compared to the I.P. injection route, with two out of three mice with I.V. injection showing marked fluorescence in the lung (FIG. 11). In one of the two mice ex vivo imaging of the lung and heart was compared to the ex vivo imaging of the lung and heart of a control mouse. The imaging data thus obtained confirm that: 1) the fluorescence detected in vivo in the thorax region was indeed coming from the lung and from the heart; and 2) since no fluorescence was detected in the lung and heart of the control mouse, the signal detected in the fibrotic mouse injected with FL4247 is very likely a result of different lung tissues induced by fibrosis. Finally, despite being studied in a pathological model (mouse pulmonary fibrosis model), the distribution of FL4247 in both routes of injection, beside the lung, followed similar patterns as observed with the biodistribution studies (FIGS. 9A and 9B and Example 8).

### REFERENCES

All patents, patent applications and publications referred to herein and throughout the application are hereby incorporated by reference.
Abou Neel et al, Day & Hyun. (2012) Collagen—Emerging Collagen-based Therapies Hit the patient. Adv. Drug Del. Rev. September 2012, in press.
Adiguzel E, Ahmad P J, Franco C, Bendeck M P. (2009) Collagens in the progression and complications of atherosclerosis. Vasc Med. 14(1), 73-89.
Andersen N H, Olsen K A, Fesinmeyer R M, Tan X, Hudson F M, Eidenschink L A, Farazi S R. (2006) Minimization and optimization of designed β-hairpin folds. J. Am. Chem. Soc. 128, 6101-6110.
Blanco F J, Rivas G, Serrano L. (1994) A short linear peptide that folds into a native stable beta-hairpin in aqueous solution. Nat Struct Biol. 1, 584-590.
Bogatkevich G S, Ludwicka-Bradley A, Nietert P J, Akter T, van Ryn J, Silver R M. (2011) Antiinflammatory and antifibrotic effects of the oral direct thrombin inhibitor dabigatran etexilate in a murine model of interstitial lung disease. Arthritis & Rheumatism. 63(5), 1416-1425.
Brinkley M. (1992) A brief survey of methods for preparing protein conjugates with dyes, haptens, and cross-linking reagents. Bioconjugate Chemistry. 3, 2-13.
Caravan P, Das B, Dumas S, Epstein F H, Helm P A, Jacques V, Koerner S, Kolodziej A, Shen L, Sun W-C, Zhang Z. (2007) Collagen-Targeted MRI Contrast Agent for Molecular Imaging of Fibrosis. Angew. Chem. 46(43), 8171-8173.
Chan J M, Zhang L, Tong R, Ghosh D, Gao W, Liao G, Yuet K P, Gray D, Rhee J W, Cheng J, Golomb G, Libby P, Langer R, Farokhzad O C. (2010) Spatiotemporal controlled delivery of nanoparticles to injured vasculature. Proc Natl Acad Sci U.S.A. 107, 2213-2218.
Chen Z, Xu P, Barbier J R, Willick G, Ni F. (2005) Solution Structure of the Osteogenic 1-31 Fragment of the Human Parathyroid Hormone. Biochem. 39, 12766-12777.
Chen F, Wang D. (2010) Novel technologies for the prevention and treatment of dental caries: a patent survey. Expert Opin Ther Pat. 20(5), 681-694.
Cochran A G, Tong R T, Starovasnik M A, Park E J, McDowell R S, Theaker J E, Skelton N J. (2001a) A minimal peptide scaffold for beta-turn display: optimizing a strand position in disulfide-cyclized beta-hairpins. J Am Chem Soc. 123(4), 625-632.
Cochran A G, Skelton N J, Starovasnik M A. (2001b) Tryptophan zippers: stable, monomeric beta-hairpins. Proc Natl Acad Sci U.S.A. 98, 5578-5583.
Cochran A G, Starovasnik M A, Skelton N. (2005) Hairpin Peptides with a Novel Structural Motif and Methods Relating Thereto. U.S. Pat. No. 6,914,123 issued Jul. 5, 2005.
Cochran A G, Starovasnik M A, Skelton N. (2007) Hairpin Peptides with a Novel Structural Motif and Methods Relating Thereto. U.S. Pat. No. 7,229,727 issued Jun. 12, 2007.
Collier J H, Segura T. (2011) Evolving the use of peptides as components in Biomaterials. Biomaterials. 32(18), 4198-4204.
Depraetere H, Viaene A, Deroo S, Vauterin S, Deckmyn H. (1998) Identification of peptides, selected by phage display technology, that inhibit von Willebrand factor binding to collagen. Blood. 92, 4207-4211.
Duncan R. (2003) The dawning era of polymer therapeutics. Nat Rev Drug Discov. 2(5), 347-360.
Eckly A, Hechler B, Freund M, Zerr M, Cazenave J-P, Lanza F, Mangin Ph, Gachet C. (2011) Mechanisms underlying FeCl₃-induced arterial thrombosis. Journal of Thrombosis and Haemostasis. 9(4), 779-789.
Fesinmeyer R M, Hudson F M, Andersen N H, (2004) Enhanced hairpin stability through loop design, the case of the protein G B1 domain hairpin. J. Am. Chem. Soc. 126, 7238-7243.
Gabriela D, Busso N, Sob A, van den Berghc H, Gurnya R, Langea N. (2009) Thrombin-sensitive photodynamic agents: A novel strategy for selective synovectomy in rheumatoid arthritis. Journal of Controlled Release. 138(3), 225-234.
Ghosh S S, Kao P M, McCue A W, Chappelle H L. (1990) Use of maleimide-thiol coupling chemistry for efficient syntheses of oligonucleotide-enzyme conjugate hybridization probes. Bioconjug Chem. 1(1), 71-6.
Han Q, Sun W, Lin H, Zhao W, Gao Y, Zhao Y, Chen B, Xiao Z, Hu W, Li Y, Yang B, Dai J. (2009) Linear ordered collagen scaffolds loaded with collagen-binding brain-derived neurotrophic factor improve the recovery of spinal cord injury in rats. Tissue Eng. Part A. 15, 2927-2935.
Helms B A, Reulen S W A, Nijhuis S, de Graaf-Heuvelmans P T H M, Merkx M, Meijer E W. (2009) High-Affinity Peptide-Based Collagen Targeting Using Synthetic Phage Mimics: From Phage Display to Dendrimer Display. J. Am. Chem. Soc. 131(33), 11683-11685.
Houdijk W P, Sakariassen K S, Nievelstein P F, Sixma J J. (1985) Role of factor VIII-von Willebrand factor and fibronectin in the interaction of platelets in flowing blood with monomeric and fibrillar human collagen types I and III. J Clin Invest. 75(2), 531-540.
Huyghues-Despointes, B M P, Qu X, Tsai J, Scholtz J M (2006) Terminal ion pairs stabilize the second β-hairpin of the B1 domain of protein G. Proteins: Struct. Funct. and Bioinformat. 63, 1005-1017.
Koga T, Oho T, Shimazaki Y, Nakano Y. (2002) Immunization against dental caries. Vaccine. 20, 2027-2044.
Lee K Y, Kong H J, Mooney D J. (2008) Quantifying interactions between cell receptors and adhesion ligand-modified polymers in solution. Macromol Biosci. 8, 140-145.
Lee K Y, Mooney D J. (2012) Alginate: properties and biomedical applications. Progress in polymer science. 37(1), 106-126.
Lett E, Gangloff S, Zimmermann M, Wachsmann D, Klein J P. (1994) Immunogenicity of polysaccharides conjugated to peptides containing T- and B-cell epitopes. Infect. Immun. 62, 785-792.
Lett E, Klopfenstein C, Klein J P, Schöller M, Wachsmann D. (1995) Mucosal immunogenicity of polysaccharides conjugated to a peptide or multiple-antigen peptide containing T- and B-cell epitopes. Infect. Immun. 63, 2645-51.
Lipshutz B H, Ghorai S. (2008) Transition metal-catalyzed cross-couplings going green: in water at room temperature. Aldrichim. Acta 41, 59-72.
Liu T, Gibbons R J. (1990) Binding of steptococci of the “mutans” group to type 1 collagen associated with apatitic surfaces. Oral Microbiol. Immunol. 5(3), 131-6.
Love R M, McMillan M D, Jenkinson H F. (1997) Invasion of dentinal tubules by oral streptococci Is aoosciated with collagen recognition mediated by the antigen I/II family of polypeptides. Infect. Immun. 65, 5157-64.
Ma J, Goldberg G I, Tjandra N. (2008) Weak alignment of biomolecules in collagen gels: an alternative way to yield residual dipolar couplings for NMR spectroscopy. J. Am. Chem. Soc. 130, 16148-16149.
Mandavi A, Ferreira L, Sundback C, Nichol J W, Chan E P, CarterDJD, Bettinger C J, Patanavanich S, Chignozha L, Ben-Joseph E, Galakatos A, Pryor H, Pomerantseva I, Masiakos P T, Faquin W, Zumbuehl A, Hong S, BorensteinJ, Vacanti J, Langer R, Karp J M. (2008) A biodegradable and biocompatible gecko-inspired tissue adhesive. PNAS. 105(7), 2307-2312.
Majumdar M, Siahaan T J. (2012) Peptide-mediated targeted drug delivery. Medicinal Research Reviews. 32(3), 637-658.
Mirassou Y, Santiveri C M, Perez de Vega M J, González-Muñiz R, Jiménez M A. (2009) Disulfide Bonds versus Trp∩∩∩Trp Pairs in Irregular â-Hairpins: NMR Structure of Vammin Loop 3-Derived Peptides as a Case Study. ChemBioChem. 10(5), 902-910.
Mo X, Iwata H, Matsuda S, Ikada Y. (2000) Soft tissue adhesive composed of modified gelatin and polysaccharides. J Biomater Sci Polym Ed. 11, 341-351.
Morris R, Winyard P G, Blake D R, Morris C J. (1994) Thrombin in inflammation and healing: relevance to rheumatoid arthritis. Ann Rheum Dis. 53(1), 72-79.
Mushero N, Gershenson A. (2011) Determining serpin conformational distributions with single molecule fluorescence. Methods Enzymol. 501, 351-377.
Muzzard J, Sarda-Mantel L, Loyau S, Mealemans A, Louedec L, Bantsimba-Malanda C, Hervatin F, Marchal-Somme J, Michel J B, Le Guludec D, Billiald P, Jandrot-Perrus M. (2009) Non-invasive molecular imaging of fibrosis using a collagen-targeted peptidomimetic of the platelet collagen receptor glycoprotein VI. PLoS one. 4 (e 5585) I-10.

Ni F, Zhu Y, Scheraga H A. (1995) Thrombin-bound Structures of Designed Analogs of Human Fibrinopeptide A Determined by Quantitative Transferred NOE Spectroscopy: A New Structural Basis for Thrombin Specificity. J. Mol. Biol. (1995) 252, 656-671.
Nikawa H, Egusa H, Yamashiro H, Nishimura M, Makihira S, Jin C, Fukushima H, Hamada T. (2006) The effect of salive or serum on bacterial and Candida albicans colonization on type I collagen. J Oral Rehab. 33, 767-774.
O'Neil C P, van der Vliesa A J, Vellutoa D, Wandreya C, Demurtasb D, Dubochetb J, Hubbell J A. 2009) Extracellular matrix binding mixed micelles for drug delivery applications. J Control Release. 137, 146-151.
Penz S, Reininger A J, Brandl R, Goyal P, Rabie T, Bernlochner I, Rother E, Goetz C, Engelmann B, Smethurst P A, Ouwehand W H, Farndale R, Nieswandt B, Siess W. (2005) Human atheromatous plaques stimulate thrombus formation by activating platelet glycoprotein VI. FASEB J. 19, 898-909.
Peppas N A, Thomas B. McGinity J. (2009) Molecular Aspects of Mucoadhesive Carrier Development for Drug Delivery and Improved Absorption. J Biomater Sci Polym Ed. 20(1), 1-20.
Peter K, Gupta A, Nordt T, Bauer S, Runge M S, Bode C. (2003) Construction and in vitro testing of a novel fab-hirudin-based fusion protein that targets fibrin and inhibits thrombin in a factor xa-dependent manner. J Cardiovasc Pharmacol. 42, 237-244.
Peters D, Kastantin M, Kotamraju V R, Karmali P P, Gujraty K, Tirrell M, Ruoslahti E. (2009) Targeting atherosclerosis by using modular, multifunctional micelles. Proc Natl Acad Sci U.S.A. 106, 9815-9819.
Petersen F C, Pasco S, Ogier J, Klein J P, Assev S, Scheie A A. (2001) Expression and functional properties of the Streptococcus intermedius surface protein antigen I/II. Infect. Immun. 69, 4647-4653.
Reininger A J, Bernlochner I, Penz S M, Ravanat C, Smethurst P, Farndale R W, Gachet C, Brandl R, Siess W. (2010) A 2-step mechanism of arterial thrombus formation induced by human atherosclerotic plaques. J Am Coll Cardiol. 55, 1147-1158.
Rivas J M, Speziale P, Patti J M, Höök M. (2004) MSCRAMM-targeted vaccines and immunotherapy for staphylococcal infection. Curr. Opin. Drug Discov. Devel. 7, 223-7.
Rooseboom M, Commandeur J N M, Vermeulen N P E. (2004) Enzyme-Catalyzed Activation of Anticancer Prodrugs. Pharmacol Rev. 56, 53-102.
Rothenfluh D A, Bermudez H, O'Neil C P, Hubbell J A. (2008) Biofunctional polymer nanoparticles for intra-articular targeting and retention in cartilage. Nature Materials. 7, 248-254.
Russel S J, Blandl T, Skelton N J, Cochran A G (2003) Stability of cyclic β-hairpins: assymmetric dontributions from side chains of a hydrogen-borded cross-strand residue pair. J Am Chem Soc. 125, 388-395.
Sanderson C J, Wilson D V. (1971) A simple method for coupling proteins to insoluble polysaccharides. Immunology. 20(6), 1061-1065.
Santiveri C M, Jiménez M A. (2010) Tryptophan residues: Scarce in proteins but strong stabilizers of â-hairpin peptides. Bioploymers: Peptide Science. 94(6), 779-790.
Sawada R, Peterson C Y, Gonzalez A M, Potenza B M, Mueller B, Coimbra R, Eliceiri B, Baird A (2011) A Phage-targeting strategy for the design of spatiotemporal drug delivery from grafted matrices. Fibrogenesis & Tissue Repair. 4:7.
Sciotti M A, Yamodo I, Klein J P, Ogier J A. (1997) The N-terminal half part of the oral steptococcal antigen I/Iif contains two distinct binding domains. FEMS Microbiol. Lett. 153(2), 439-45.
Setton L. (2008) Polymer therapeutics: Reservoir drugs. Nature Materials. 7, 172-174.
Sukhova G K, Schönbeck U, Rabkin E, Schoen F J, Robin Poole A R, Billinghurst R C, Libby P. (1999) Evidence for Increased Collagenolysis by Interstitial Collagenases-1 and -3 in Vulnerable Human Atheromatous Plaques. Circulation. 99, 2503-2509.
Sun W, Sun C, Lin H, Zhao H, Wang J, Ma H, Chen B, Xiao Z, Dai J. (2009) The effect of collagen-binding NGF-beta on the promotion of sciatic nerve regeneration in a rat sciatic nerve crush injury model. Biomaterials. 30(27), 4649-4656.
Switalski L M, Butcher W G, Caufield P C, Lantz M S. (1993) Collagen mediates adhesion of Streptococcus mutans to human dentin. Infect. Immun. 61, 4119-4125.
Takagi J, Asai H, Saito Y. (1992) A collagen/gelatin-binding decapeptide derived from bovine propolypeptide of von Willebrand factor. Biochemistry. 31(36), 8530-8534.
Takekiyo T, Wu L, Yoshimura Y, Shimizu A, Keiderling T A. (2009) Relationship between hydrophobic interactions and secondary structure stability for trpzip β-hairpin peptides. Biochemistry 48, 1542-1552.
Topcic D, Kim W, Holien J K, Jia F, Armstrong P C, Hohmann J D, Straub A, Krippner G, Haller C A, Domeij H, Hagemeyer C E, Parker M W, Chaikof E L, Peter K. (2011) An Activation-Specific Platelet Inhibitor That Can Be Turned On/Off by Medically Used Hypothermia. Arterioscler Thromb Vasc Biol. 31, 2015-2023.
Torbet J, Malbouyres M, Builles N, Justin V, Roulet M, Damour O, Oldberg A, Ruggiero F, Hulmes D J S. (2007) Orthogonal scaffold of magnetically aligned collagen lamellae for corneal stroma reconstruction. Biomaterials. 28, 4268-4278.
Vanhoorelbeke K, Depraetere H, Romijn R A P, Huizinga E G, Demaeyer M, Deckmyn H. (2003) A consensus tetra-peptide selected by phage display adapts the conformation of a dominant discontinuous epitope of a monoclonal anti-VWF antibody that inhibits the in vivo VWF-collagen interaction. J Biol Chem. 278, 37815-37821.
Wang X, Xu L. (2005) An optimized murine model of ferric chloride-induced arterial thrombosis for thrombosis research. Thromb. Res. 115, 95-100.
Wang D A, Varghese S, Sharma B, Strehin I, Fermanian S, Fairbrother D H, Cascio B, Elisseeff J H. (2007) Multifunctional chondroitin sulphate for cartilage tissue-biomaterial integration. Nat. Mater. 6, 385-392.
Wu L, McElheny D, Huang R, Keiderling T A. (2009) Role of tryptophan-tryptophan interactions in trpzip β-hairpin-formation, structure and stablity. Biochemistry 48, 10362-10371.
Wynn T A. (2004) Fibrotic disease and the T(H)1/T(H)2 paradigm. Nat. Rev. Immunol. 4, 583-594.
Zhang Z, Tan S, Feng S S. (2012) Vitamin E TPGS as a molecular biomaterial for drug delivery. Biomaterials. 33, 4889-4906.
Zhu T, Mo H, Wang N, Nam D S, Cao Y, Koup R A, Ho D D. (1993) Genotypic and phenotypic characterization of HIV-1 in patients with primary infections. Science. 281, 1179-1181.
WO/2012/142696
WO 95/04069
WO/2004/076670

1. A molecule of Formula (I)
(Y)ₙ-(CBLH)-(Z)ₘ (I)
that specifically binds to collagen where:
Y is a first compound of interest;
Z is a second compound of interest;
Y and Z may be different or the same, and n and m are independently 0 or 1 with the proviso that at least one of n and m is 1; and,
CBLH is a collagen-binding linear hairpin peptide comprising 19 or fewer amino acids and comprising a turn amino acid sequence comprising 4 to 6 amino acid residues providing a stable turn structure, the turn sequence flanked on one side by a first flanking sequence comprising SEQ ID NO: 1 and flanked on the other side by a second flanking sequence comprising SEQ ID NO: 2, the W residue at position 1 of SEQ ID NO: 1 forming a cross-strand indole-indole or cation-π interaction pair with the amino acid residue at position 3 of SEQ ID NO: 2 without any disulfide bond.

2. The molecule according to claim 1, wherein the amino acid residue at position 2 of SEQ ID NO: 1 is threonine.

3. The molecule according to claim 1, wherein the amino acid residue at position 3 of SEQ ID NO: 1 is tryptophan or tyrosine.

4. The molecule according to claim 1, wherein the amino acid residue at position 2 of SEQ ID NO: 2 is threonine.

5. The molecule according to claim 1, wherein the amino acid residue at position 3 of SEQ ID NO: 2 is tryptophan or arginine.

6. The molecule according to claim 1, wherein collagen-binding linear hairpin peptide comprises SEQ ID NO: 15, SEQ ID NO: 16, SEQ ID NO: 21, SEQ ID NO: 22, SEQ ID NO: 23, SEQ ID NO: 26, SEQ ID NO: 27, SEQ ID NO: 30, SEQ ID NO: 31, SEQ ID NO: 32, SEQ ID NO: 33, SEQ ID NO: 34, SEQ ID NO: 35.

7. The molecule according to claim 1, wherein collagen-binding linear hairpin peptide comprises SEQ ID NO: 17 SEQ ID NO: 18, SEQ ID NO: 19, SEQ ID NO: 20, SEQ ID NO: 24, SEQ ID NO: 25, SEQ ID NO: 28, SEQ ID NO: 29, SEQ ID NO: 36, SEQ ID NO: 37, SEQ ID NO: 38, SEQ ID NO: 39, SEQ ID NO: 40 or SEQ ID NO: 41.

8. The molecule according to claim 1, wherein Y is KGG, acetyl, SEQ ID NO: 53, SEQ ID NO: 54, CGG, G, alginate-COOH, Dextran-COOH, or Dextran-NH2 or D-α-tocopheryl polyethylene glycol succinate (TPGS).

9. The molecule according to claim 1, wherein Z is GGK, SEQ ID NO: 55, SEQ ID NO: 56, SEQ ID NO: 57, GGC, G, Fluor750, alginate-COOH, Dextran-COOH, Dextran-NH2, D-α-tocopheryl polyethylene glycol succinate (TPGS), SEQ ID NO: 58, SEQ ID NO: 59, SEQ ID NO: 60, SEQ ID NO:110, SEQ ID NO: 61, SEQ ID NO: 62 or SEQ ID NO: 63.

10. The molecule according to claim 1, wherein the molecule of Formula (I) is SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO:111, SEQ ID NO: 89, SEQ ID NO: 90, SEQ ID NO: 91, SEQ ID NO: 105 or SEQ ID NO:106.

11. A method of delivering a compound of interest to a site of interest, the site of interest containing collagen, the method comprising providing a molecule according to claim 1 at the site of interest, whereby the collagen-binding linear hairpin peptide binds to collagen at the site of interest thereby delivering the compound of interest to the site of interest.

12. The method according to claim 11, wherein the site of interest is fibrotic or fibrosing tissue.

13. A pharmaceutical composition comprising a molecule as defined in claim 1 and a pharmaceutically acceptable carrier, diluent or excipient.


SEQUENCE LISTING (111 sequences)

SEQ ID NO: 1 - protein, 3 aa, Artificial Sequence
  First flanking sequence
WXX

SEQ ID NO: 2 - protein, 3 aa, Artificial Sequence
  Second flanking sequence
XXX

SEQ ID NO: 3 - protein, 6 aa, Artificial Sequence
  Turn sequence
DDATKT

SEQ ID NO: 4 - protein, 4 aa, Artificial Sequence
  Turn sequence
EPNK

SEQ ID NO: 5 - protein, 4 aa, Artificial Sequence
  Turn sequence
ENGK

SEQ ID NO: 6 - protein, 4 aa, Artificial Sequence
  Turn sequence
EGNK

SEQ ID NO: 7 - protein, 4 aa, Artificial Sequence
  Turn sequence
NGSA

SEQ ID NO: 8 - protein, 4 aa, Artificial Sequence
  Turn sequence
NGTN

SEQ ID NO: 9 - protein, 5 aa, Artificial Sequence
  Turn sequence
NGSTA

SEQ ID NO: 10 - protein, 4 aa, Artificial Sequence
  Turn sequence
NDSN

SEQ ID NO: 11 - protein, 4 aa, Artificial Sequence
  Turn sequence
NNSA

SEQ ID NO: 12 - protein, 4 aa, Artificial Sequence
  Turn sequence
NNST

SEQ ID NO: 13 - protein, 4 aa, Artificial Sequence
  Turn sequence
NGSN

SEQ ID NO: 14 - protein, 6 aa, Artificial Sequence
  Turn sequence
NPATGK

SEQ ID NO: 15 - protein, 16 aa, Artificial Sequence
  CBLH peptide - trpzip6
GEWTWDDATKTWTVTE

SEQ ID NO: 16 - protein, 16 aa, Artificial Sequence
  CBLH peptide - trpzip5
GEWTYDDATKTFTWTE

SEQ ID NO: 17 - protein, 16 aa, Artificial Sequence
  CBLH peptide - trpzip4
GEWTWDDATKTWTWTE

SEQ ID NO: 18 - protein, 12 aa, Artificial Sequence
  CBLH peptide - trpzip3
SWTWEPNKWTWK

SEQ ID NO: 19 - protein, 12 aa, Artificial Sequence
  CBLH peptide - trpzip2
SWTWENGKWTWK

SEQ ID NO: 20 - protein, 12 aa, Artificial Sequence
  CBLH peptide - trpzip1
SWTWEGNKWTWK

SEQ ID NO: 21 - protein, 12 aa, Artificial Sequence
  CBLH peptide - Y4-trpzip1
SWTYEGNKWTWK

SEQ ID NO: 22 - protein, 12 aa, Artificial Sequence
  CBLH peptide - Y4Y9-trpzip1
SWTYEGNKYTWK

SEQ ID NO: 23 - protein, 12 aa, Artificial Sequence
  CBLH peptide - WYYW-trpzip2
SWTYENGKYTWK

SEQ ID NO: 24 - protein, 12 aa, Artificial Sequence
  CBLH peptide - v4-gp120
TWTWNGSAWTWN

SEQ ID NO: 25 - protein, 14 aa, Artificial Sequence
  CBLH peptide - v4-gp120-ext
STWTWNGSAWTWNE

SEQ ID NO: 26 - protein, 12 aa, Artificial Sequence
  CBLH peptide - v4-gp160
TWTWNGTNWTRN

SEQ ID NO: 27 - protein, 14 aa, Artificial Sequence
  CBLH peptide - v4-gp160-ext
STWTWNGTNWTRND

SEQ ID NO: 28 - protein, 13 aa, Artificial Sequence
  CBLH peptide - pp-D7EUT8
TWRWNGSTAWTWS

SEQ ID NO: 29 - protein, 17 aa, Artificial Sequence
  CBLH peptide - pp-D7EUT8-ext
STWRWNGSTAWTWSTAS

SEQ ID NO: 30 - protein, 12 aa, Artificial Sequence
  CBLH peptide - v4-gp120v2
TWTRNDSNWTWN

SEQ ID NO: 31 - protein, 14 aa, Artificial Sequence
  CBLH peptide - v4-gp120v2-ext
STWTRNDSNWTWNG

SEQ ID NO: 32 - protein, 12 aa, Artificial Sequence
  CBLH peptide - pp-Q77413
TWTRNNSAWTWN

SEQ ID NO: 33 - protein, 14 aa, Artificial Sequence
  CBLH peptide - pp-Q77413-ext
STWTRNNSAWTWNG

SEQ ID NO: 34 - protein, 12 aa, Artificial Sequence
  CBLH peptide - pp-Q77425
TWTRNNSTWTWN

SEQ ID NO: 35 - protein, 14 aa, Artificial Sequence
  CBLH peptide - pp-Q77425-ext
STWTRNNSTWTWNG

SEQ ID NO: 36 - protein, 12 aa, Artificial Sequence
  CBLH peptide - pp-Q904S5
TWAWNGSNWTWN

SEQ ID NO: 37 - protein, 14 aa, Artificial Sequence
  CBLH peptide - pp-Q904S5-ext
STWAWNGSNWTWNG

SEQ ID NO: 38 - protein, 16 aa, Artificial Sequence
  CBLH peptide - trpzip7
GEWHWDDATKTWVWTE

SEQ ID NO: 39 - protein, 16 aa, Artificial Sequence
  CBLH peptide - trpzip8
GEWVWDDATKTWHWTE

SEQ ID NO: 40 - protein, 16 aa, Artificial Sequence
  CBLH peptide - trpzip9
GEWVWDDATKTWVWTE

SEQ ID NO: 41 - protein, 16 aa, Artificial Sequence
  CBLH peptide - HP5W4
KKWTWNPATGKWTWQE

SEQ ID NO: 42 - protein, 10 aa, Artificial Sequence
  III-3 - collagen binding peptide no hairpin
WREPSFCALS

SEQ ID NO: 43 - protein, 8 aa, Artificial Sequence
  Q-peptide - collagen binding peptide no hairpin
CVWLWEQC

SEQ ID NO: 44 - protein, 8 aa, Artificial Sequence
  N-peptide - collagen binding peptide no hairpin
CVWLWENC

SEQ ID NO: 45 - protein, 8 aa, Artificial Sequence
  C6H5 - collagen binding peptide no hairpin
CMTSPWRC

SEQ ID NO: 46 - protein, 8 aa, Artificial Sequence
  C6G12 - collagen binding peptide no hairpin
CRTSPWRC

SEQ ID NO: 47 - protein, 8 aa, Artificial Sequence
  C6A12 - collagen binding peptide no hairpin
CYRSPWRC

SEQ ID NO: 48 - protein, 16 aa, Artificial Sequence
  EP-3533p - collagen binding peptide no hairpin
GKWHCTTKFPHHYCLY

SEQ ID NO: 49 - protein, 6 aa, Artificial Sequence
  C1-3 - collagen binding peptide no hairpin
WYRGRL

SEQ ID NO: 50 - protein, 7 aa, Artificial Sequence
  Helms 2009 - collagen binding peptide no hairpin
HVWMQAP

SEQ ID NO: 51 - protein, 18 aa, Artificial Sequence
  Muzzard 2009 - collagen binding peptide no hairpin
CPGRVMHGLHLGDDEGPC

SEQ ID NO: 52 - protein, 7 aa, Artificial Sequence
  C-11 - collagen binding peptide no hairpin
KLWLLPK

SEQ ID NO: 53 - protein, 5 aa, Artificial Sequence
  Thrombin inhibitor
IRFTD

SEQ ID NO: 54 - protein, 9 aa, Artificial Sequence
  Y moiety
FQPRPRFTD

SEQ ID NO: 55 - protein, 11 aa, Artificial Sequence
  Z moiety
GDFEEIPEEYL

SEQ ID NO: 56 - protein, 12 aa, Artificial Sequence
  Z moiety
GDFEEIPEEYLQ

SEQ ID NO: 57 - protein, 8 aa, Artificial Sequence
  Z moiety
GSFNPRGS

SEQ ID NO: 58 - protein, 132 aa, Artificial Sequence
  GGS-VHpro10 - Z moiety
GGSDVQLQASGGGLVQAGGSLRLTCAASGRTFDRYGWFRQAPGKEREFVASIGTRLHYADSVKGRFTISRDNAKSTAFLEMNSLKPEDTAVYYCAAAESTRNWYYKMSNDYDYWGQGTQVTVSSLEHHHHHH

SEQ ID NO: 59 - protein, 134 aa, Artificial Sequence
  GDFEE-VHpro10 - Z moiety
GDFEEDVQLQASGGGLVQAGGSLRLTCAASGRTFDRYGWFRQAPGKEREFVASIGTRLHYADSVKGRFTISRDNAKSTAFLEMNSLKPEDTAVYYCAAAESTRNWYYKMSNDYDYWGQGTQVTVSSLEHHHHHH

SEQ ID NO: 60 - protein, 135 aa, Artificial Sequence
  GGGGS-VHpro10 - Z moiety
GGGGSSDVQLQASGGGLVQAGGSLRLTCAASGRTFDRYGWFRQAPGKEREFVASIGTRLHYADSVKGRFTISRDNAKSTAFLEMNSLKPEDTAVYYCAAAESTRNWYYKMSNDYDYWGQGTQVTVSSLEHHHHHH

SEQ ID NO: 61 - protein, 137 aa, Artificial Sequence
  GGS-VHpro5 - Z moiety
GGSDVQLQASGGGLVQAGGSLRLTCAASGRTFSSLSIAWFRQAPGKEREFVAGIRWTAGSKTYANWVKGRFTISRDNAKSTAFLEMNSLKPEDTAVYYCAADNISDWGISKQLRTYHYWGQGTQVTVSSLEHHHHHH

SEQ ID NO: 62 - protein, 141 aa, Artificial Sequence
  GGS-VHpro10c - Z moiety
GGSCHNDGGGGSDVQLQASGGGLVQAGGSLRLTCAASGRTFDRYGWFRQAPGKEREFVASIGTRLHYADSVKGRFTISRDNAKSTAFLEMNSLKPEDTAVYYCAAAESTRNWYYKMSNDYDYWGQGTQVTVSSLEHHHHHH

SEQ ID NO: 63 - protein, 146 aa, Artificial Sequence
  GGS-VHpro5c - Z moiety
GGSCHNDGGGGSDVQLQASGGGLVQAGGSLRLTCAASGRTFSSLSIAWFRQAPGKEREFVAGIRWTAGSKTYANWVKGRFTISRDNAKSTAFLEMNSLKPEDTAVYYCAADNISDWGISKQLRTYHYWGQGTQVTVSSLEHHHHHH

SEQ ID NO: 64 - protein, 129 aa, Artificial Sequence
  Artificial VHpro10
DVQLQASGGGLVQAGGSLRLTCAASGRTFDRYGWFRQAPGKEREFVASIGTRLHYADSVKGRFTISRDNAKSTAFLEMNSLKPEDTAVYYCAAAESTRNWYYKMSNDYDYWGQGTQVTVSSLEHHHHHH

SEQ ID NO: 65 - protein, 134 aa, Artificial Sequence
  Artificial VHpro5
DVQLQASGGGLVQAGGSLRLTCAASGRTFSSLSIAWFRQAPGKEREFVAGIRWTAGSKTYANWVKGRFTISRDNAKSTAFLEMNSLKPEDTAVYYCAADNISDWGISKQLRTYHYWGQGTQVTVSSLEHHHHHH

SEQ ID NO: 66 - protein, 138 aa, Artificial Sequence
  VHpro10c
CHNDGGGGSDVQLQASGGGLVQAGGSLRLTCAASGRTFDRYGWFRQAPGKEREFVASIGTRLHYADSVKGRFTISRDNAKSTAFLEMNSLKPEDTAVYYCAAAESTRNWYYKMSNDYDYWGQGTQVTVSSLEHHHHHH

SEQ ID NO: 67 - protein, 143 aa, Artificial Sequence
  VHpro5c
CHNDGGGGSDVQLQASGGGLVQAGGSLRLTCAASGRTFSSLSIAWFRQAPGKEREFVAGIRWTAGSKTYANWVKGRFTISRDNAKSTAFLEMNSLKPEDTAVYYCAADNISDWGISKQLRTYHYWGQGTQVTVSSLEHHHHHH

SEQ ID NO: 68 - protein, 13 aa, Artificial Sequence
  MH2-wZIP4-F1 - does not bind collagen
IRFTDGEWTWDDA

SEQ ID NO: 69 - protein, 8 aa, Artificial Sequence
  MH2-wZIP4-F2 - does not bind collagen
GEWTWDDA

SEQ ID NO: 70 - protein, 16 aa, Artificial Sequence
  gb1 - does not bind collagen
GEWTYDDATKTFTVTE

SEQ ID NO: 71 - protein, 12 aa, Artificial Sequence
  HP7 - does not bind collagen
KTWNPATGKWTE

SEQ ID NO: 72 - protein, 12 aa, Artificial Sequence
  Y2Y11-trpzip1 - does not bind collagen
SYTWEGNKWTYK

SEQ ID NO: 73 - protein, 12 aa, Artificial Sequence
  W4W9-trpzip2 - does not bind collagen
SVTWENGKWTVK

SEQ ID NO: 74 - protein, 12 aa, Artificial Sequence
  W2W11-trpzip2 - does not bind collagen
SWTVENGKVTWK

SEQ ID NO: 75 - protein, 12 aa, Artificial Sequence
  YWWY-trpzip2 - does not bind collagen
SYTWENGKWTYK

SEQ ID NO: 76 - protein, 17 aa, Artificial Sequence
  v4-gp120-ext2 - molecule of Formula (I)
STWTWNGSAWTWNEGGK

SEQ ID NO: 77 - protein, 17 aa, Artificial Sequence
  v4-gp160-ext2 - molecule of Formula (I)
STWTWNGTNWTRNDGGK

SEQ ID NO: 78 - protein, 30 aa, Artificial Sequence
  MH2-wZIP1 - molecule of Formula (I)
IRFTDGSWTWEGNKWTWKGDFEEIPEEYLQ

SEQ ID NO: 79 - protein, 32 aa, Artificial Sequence
  MH2-wZIP1GG - molecule of Formula (I)
IRFTDGGSWTWEGNKWTWKGGDFEEIPEEYLQ

SEQ ID NO: 80 - protein, 33 aa, Artificial Sequence
  MH2-wZIP4 - molecule of Formula (I)
IRFDTGEWTWDDATKTWTWTEGDFEEIPEEYLQ

SEQ ID NO: 81 - protein, 27 aa, Artificial Sequence
  MH2-wZIP4-F3 - molecule of Formula (I)
GEWTWDDATKTWTWTEGDFEEIPEEYL

SEQ ID NO: 82 - protein, 29 aa, Artificial Sequence
  MH2-wZIP4-F4 - molecule of Formula (I)
IRFTDGEWTWDDATKTWTWTEGSFNPRGS

SEQ ID NO: 83 - protein, 24 aa, Artificial Sequence
  MH2-wZIP4-F5 - molecule of Formula (I)
IRFTDGEWTWDDATKTWTWTEGGC

SEQ ID NO: 84 - protein, 22 aa, Artificial Sequence
  MH2-wZIP4-F6 - molecule of Formula (I)
IRFTDGEWTWDDATKTWTWTEG

SEQ ID NO: 85 - protein, 24 aa, Artificial Sequence
  FL4247 - molecule of Formula (I)
IRFTDGEWTWDDATKTWTWTEGGC

SEQ ID NO: 86 - protein, 153 aa, Artificial Sequence
  WZ4-GGS-VHpro10 - molecule of Formula (I)
IRFTDGEWTWDDATKTWTWTEGGSDVQLQASGGGLVQAGGSLRLTCAASGRTFDRYGWFRQAPGKEREFVASIGTRLHYADSVKGRFTISRDNAKSTAFLEMNSLKPEDTAVYYCAAAESTRNWYYKMSNDYDYWGQGTQVTVSSLEHHHHHH

SEQ ID NO: 87 - protein, 155 aa, Artificial Sequence
  WZ4-GDFEE-VHpro10 - molecule of Formula (I)
IRFTDGEWTWDDATKTWTWTEGDFEEDVQLQASGGGLVQAGGSLRLTCAASGRTFDRYGWFRQAPGKEREFVASIGTRLHYADSVKGRFTISRDNAKSTAFLEMNSLKPEDTAVYYCAAAESTRNWYYKMSNDYDYWGQGTQVTVSSLEHHHHHH

SEQ ID NO: 88 - protein, 156 aa, Artificial Sequence
  WZ4-GGGGS-VHpro10 - molecule of Formula (I)
IRFTDGEWTWDDATKTWTWTEGGGGSSDVQLQASGGGLVQAGGSLRLTCAASGRTFDRYGWFRQAPGKEREFVASIGTRLHYADSVKGRFTISRDNAKSTAFLEMNSLKPEDTAVYYCAAAESTRNWYYKMSNDYDYWGQGTQVTVSSLEHHHHHH

SEQ ID NO: 89 - protein, 158 aa, Artificial Sequence
  WZ4-GGS-VHpro5 - molecule of Formula (I)
IRFTDGEWTWDDATKTWTWTEGGSDVQLQASGGGLVQAGGSLRLTCAASGRTFSSLSIAWFRQAPGKEREFVAGIRWTAGSKTYANWVKGRFTISRDNAKSTAFLEMNSLKPEDTAVYYCAADNISDWGISKQLRTYHYWGQGTQVTVSSLEHHHHHH

SEQ ID NO: 90 - protein, 37 aa, Artificial Sequence
  N-terminally extended derivative of MH2-wZIP4 - molecule of Formula (I)
FQPRPRFTDGEWTWDDATKTWTWTEGDFEEIPEEYLQ

SEQ ID NO: 91 - protein, 155 aa, Artificial Sequence
  BRI-T404 - molecule of Formula (I)
IRFTDGEWTWDDATKTWTWTEGGSEVQLQASGGGLVQSGDSLRLSCAASGRTFSTYAMGWFRQAPGKLREFVGVISSSGYTHYTNSVRGRFTISRDNAKNMVYLQMNSLKPEDTAVYYCAAADRRFIATDGKQYDYWGQGTQVTVSSLEHHHHHH

SEQ ID NO: 92 - protein, 10 aa, Artificial Sequence
  Peptide fragment
DFEEIPEEYL

SEQ ID NO: 93 - protein, 5 aa, Artificial Sequence
  Recognition site for thrombin-specific cleavage
SFNPR

SEQ ID NO: 94 - protein, 4 aa, Artificial Sequence
  Thrombin-specific peptide sequence
FNPR

SEQ ID NO: 95 - protein, 6 aa, Artificial Sequence
  Thrombin-specific recognition site
FNPRGS

SEQ ID NO: 96 - protein, 4 aa, Artificial Sequence
  Linker
GGGG

SEQ ID NO: 97 - protein, 16 aa, Artificial Sequence
  W12W14-gb1
GEWTYDDATKTWTWTE

SEQ ID NO: 98 - protein, 16 aa, Artificial Sequence
  W5W12R14-gb1
GEWTWDDATKTWTRTE

SEQ ID NO: 99 - protein, 16 aa, Artificial Sequence
  W12W14-gb1-AL
GEWTYNPATGKWTWTE

SEQ ID NO: 100 - protein, 16 aa, Artificial Sequence
  W5W12R14-gb1-AL
GEWTWNPATGKWTRTE

SEQ ID NO: 101 - protein, 16 aa, Artificial Sequence
  K1W12W14-gb1
KEWTYDDATKTWTWTE

SEQ ID NO: 102 - protein, 16 aa, Artificial Sequence
  K1W5W12R14-gb1
KEWTWDDATKTWTRTE

SEQ ID NO: 103 - protein, 15 aa, Artificial Sequence
  K1W12W14-gb1-AL
EWTYNPATGKWTWTE

SEQ ID NO: 104 - protein, 16 aa, Artificial Sequence
  K1W5W12R14-gb1-AL
KEWTWNPATGKWTRTE

SEQ ID NO: 105 - protein, 17 aa, Artificial Sequence
  CBLH5-GGC (P4447)
STWTWNGTNWTRNDGGC

SEQ ID NO: 106 - protein, 17 aa, Artificial Sequence
  FL4447
STWTWNGTNWTRNDGGC

SEQ ID NO: 107 - protein, 5 aa, Artificial Sequence
  Linker
GGGSS

SEQ ID NO: 108 - protein, 6 aa, Artificial Sequence
  Linker
GGGGSS

SEQ ID NO: 109 - protein, 5 aa, Artificial Sequence
  Linker
GDFEE

SEQ ID NO: 110 - protein, 134 aa, Artificial sequence
  GGGSS-VHpro10
GGGSSDVQLQASGGGLVQAGGSLRLTCAASGRTFDRYGWFRQAPGKEREFVASIGTRLHYADSVKGRFTISRDNAKSTAFLEMNSLKPEDTAVYYCAAAESTRNWYYKMSNDYDYWGQGTQVTVSSLEHHHHHH

SEQ ID NO: 111 - protein, 155 aa, Artificial sequence
  BRI-TP404b or WZ4-GGGSS-VHpro10
IRFTDGEWTWDDATKTWTWTEGGGSSDVQLQASGGGLVQAGGSLRLTCAASGRTFDRYGWFRQAPGKEREFVASIGTRLHYADSVKGRFTISRDNAKSTAFLEMNSLKPEDTAVYYCAAAESTRNWYYKMSNDYDYWGQGTQVTVSSLEHHHHHH